(19) United States
(12) Patent Application Publication
(10) Pub. No.: US 2016/0311806 A1
(43) Pub. Date: Oct. 27, 2016

(54) 4,4'-BIPHENYLDIYL-BIS-(1 H-IMIDAZOLYL) DERIVATIVES AS HEPATITIS HCV INHIBITORS

(71) Applicant: BRISTOL-MYERS SQUIBB COMPANY, Princeton, NJ (US)

(72) Inventors: John A. Bender, Middletown, CT (US); Zhong Yang, Southington, CT (US); Makonen Belema, North Haven, CT (US)

(21) Appl. No.: 15/100,681

(22) PCT Filed: Dec. 2, 2014
(86) PCT No.: PCT/US2014/068020

§ 371 Date: Jun. 1, 2016

Related U.S. Application Data
(60) Provisional application No. 61/914,716, filed on Dec. 11, 2013.

(51) Int. Cl. C07D 405/14; A61K 45/06; A61K 31/4178

(52) U.S. Cl. CPC C07D 405/14; A61K 31/4178; A61K 45/06

(57) ABSTRACT

The present disclosure relates to compounds of formula (I), pharmaceutical compositions and use thereof in treatment of hepatitis C virus (HCV) infection.

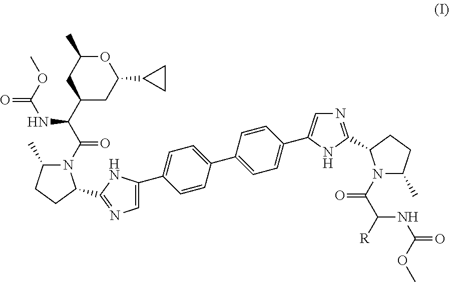

### CROSS-REFERENCE TO RELATED APPLICATIONS

This application claims priority to Provisional patent application U.S. Ser. No. 61/914,716 filed Dec. 11, 2013, hereby incorporated by reference in its entirety.

The present disclosure is generally directed to antiviral compounds, and more specifically directed to compounds which can inhibit the function of the NS5A protein encoded by Hepatitis C virus (HCV), compositions comprising such compounds, and methods for inhibiting the function of the NS5A protein.

HCV is a major human pathogen, infecting an estimated 170 million persons worldwide—roughly five times the number infected by human immunodeficiency virus type 1. A substantial fraction of these HCV infected individuals develop serious progressive liver disease, including cirrhosis and hepatocellular carcinoma.

The current standard of care for HCV, which employs a combination of pegylated-interferon and ribavirin, has a non-optimal success rate in achieving sustained viral response and causes numerous side effects. Thus, there is a clear and long-felt need to develop effective therapies to address this undermet medical need.

HCV is a positive-stranded RNA virus. Based on a comparison of the deduced amino acid sequence and the extensive similarity in the 5′ untranslated region, HCV has been classified as a separate genus in the Flaviviridae family. All members of the Flaviviridae family have enveloped virions that contain a positive stranded RNA genome encoding all known virus-specific proteins via translation of a single, uninterrupted, open reading frame.

Considerable heterogeneity is found within the nucleotide and encoded amino acid sequence throughout the HCV genome due to the high error rate of the encoded RNA dependent RNA polymerase which lacks a proof-reading capability. At least six major genotypes have been characterized, and more than 50 subtypes have been described with distribution worldwide. The clinical significance of the genetic heterogeneity of HCV has demonstrated a propensity for mutations to arise during monotherapy treatment, thus additional treatment options for use are desired. The possible modulator effect of genotypes on pathogenesis and therapy remains elusive.

The single strand HCV RNA genome is approximately 9500 nucleotides in length and has a single open reading frame (ORF) encoding a single large polyprotein of about 3000 amino acids. In infected cells, this polyprotein is cleaved at multiple sites by cellular and viral proteases to produce the structural and non-structural (NS) proteins. In the case of HCV, the generation of mature non-structural proteins (NS2, NS3, NS4A, NS4B, NS5A, and NS5B) is effected by two viral proteases. The first one is believed to be a metalloprotease and cleaves at the NS2-NS3 junction; the second one is a serine protease contained within the N-terminal region of NS3 (also referred to herein as NS3 protease) and mediates all the subsequent cleavages downstream of NS3, both in cis, at the NS3-NS4A cleavage site, and in trans, for the remaining NS4A-NS4B, NS4B-NS5A, NS5A-NS5B sites. The NS4A protein appears to serve multiple functions by both acting as a cofactor for the NS3 protease and assisting in the membrane localization of NS3 and other viral replicase components. The formation of a NS3-NS4A complex is necessary for proper protease activity resulting in increased proteolytic efficiency of the cleavage events. The NS3 protein also exhibits nucleoside triphosphatase and RNA helicase activities. NS5B (also referred to herein as HCV polymerase) is a RNA-dependent RNA polymerase that is involved in the replication of HCV genome with other HCV proteins, including NS5A, in a replicase complex.

Compounds useful for treating HCV-infected patients are desired which selectively inhibit HCV viral replication. In particular, compounds which are effective to inhibit the function of the NS5A protein are desired. The HCV NS5A protein is described, for example, in the following references: S. L. Tan, et al., Virology, 284:1-12 (2001); K.-J. Park, et al., J. Biol. Chem., 30711-30718 (2003); T. L. Tellinghuisen, et al., Nature, 435, 374 (2005); R. A. Love, et al., J. Virol, 83, 4395 (2009); N. Appel, et al., J. Biol. Chem., 281, 9833 (2006); L. Huang, J. Biol. Chem., 280, 36417 (2005); C. Rice, et al., WO2006093867.

Bachand, et. al. in WO2008/021927, published Feb. 21, 2008, disclose a series of biphenyl compounds which are useful for the treatment of Hepatitis C virus. The novel compounds of the present disclosure fall within the definition of the Formula in WO2008/021927 and are not disclosed or described by Bachand, et al. Surprisingly, it has been discovered that these compounds possess unique attributes which make them useful for the treatment of Hepatitis C virus.

In a first aspect the present disclosure provides a compound of formula (I)

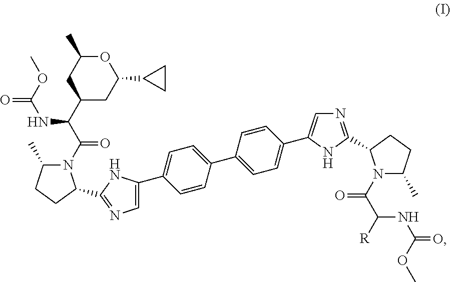

or a pharmaceutically acceptable salt thereof, wherein:

R is selected from isopropyl, phenyl, and

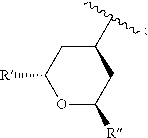

wherein R′ is selected from ethyl and methyl; and
R″ is selected from hydrogen and methyl.

In a second aspect the present disclosure provides a composition comprising a compound of formula (I), or a pharmaceutically acceptable salt thereof, and a pharmaceutically acceptable carrier. In a first embodiment of the second aspect the composition further comprises one, two, or three additional compounds having anti-HCV activity. In a second embodiment of the second aspect at least one of the additional compounds is an interferon or a ribavirin. In a third embodiment the interferon is selected from interferon alpha 2B, pegylated interferon alpha, consensus interferon, interferon alpha 2A, interferon lambda, and lymphoblastoid interferon tau.

In a fourth embodiment of the second aspect the present disclosure provides a composition comprising a compound of formula (I), or a pharmaceutically acceptable salt thereof, a pharmaceutically acceptable carrier, and one or two additional compounds having anti-HCV activity, wherein at least one of the additional compounds is selected from interleukin 2, interleukin 6, interleukin 12, a compound that enhances the development of a type 1 helper T cell response, interfering RNA, anti-sense RNA, Imiquimod, ribavirin, an inosine 5′-monophosphate dehydrogenase inhibitor, amantadine, and rimantadine.

In a fifth embodiment of the second aspect the present disclosure provides a composition comprising a compound of formula (I), or a pharmaceutically acceptable salt thereof, a pharmaceutically acceptable carrier, and one or two additional compounds having anti-HCV activity, wherein at least one of the additional compounds is effective to inhibit the function of a target selected from HCV metalloprotease, HCV serine protease, HCV polymerase, HCV helicase, HCV NS4B protein, HCV entry, HCV assembly, HCV egress, HCV NS5A protein, and IMPDH for the treatment of an HCV infection.

In a third aspect the present disclosure provides a method of treating an HCV infection in a patient, comprising administering to the patient a therapeutically effective amount of a compound of formula (I), or a pharmaceutically acceptable salt thereof. In a first embodiment of the third aspect the method further comprises administering one, two, or three additional compounds having anti-HCV activity prior to, after or simultaneously with the compound of formula (I), or a pharmaceutically acceptable salt thereof. In a second embodiment of the third aspect at least one of the additional compounds is an interferon or a ribavirin. In a third embodiment of the third aspect the interferon is selected from interferon alpha 2B, pegylated interferon alpha, consensus interferon, interferon alpha 2A, interferon lambda, and lymphoblastoid interferon tau.

In a fourth embodiment of the third aspect the present disclosure provides a method of treating an HCV infection in a patient, comprising administering to the patient a therapeutically effective amount of a compound of formula (I), or a pharmaceutically acceptable salt thereof, and one or two additional compounds having anti-HCV activity prior to, after or simultaneously with the compound of formula (I), or a pharmaceutically acceptable salt thereof, wherein at least one of the additional compounds is selected from interleukin 2, interleukin 6, interleukin 12, a compound that enhances the development of a type 1 helper T cell response, interfering RNA, anti-sense RNA, Imiquimod, ribavirin, an inosine 5′-monophosphate dehydrogenase inhibitor, amantadine, and rimantadine.

In a fifth embodiment of the third aspect the present disclosure provides a method of treating an HCV infection in a patient, comprising administering to the patient a therapeutically effective amount of a compound of formula (I), or a pharmaceutically acceptable salt thereof, and one or two additional compounds having anti-HCV activity prior to, after or simultaneously with the compound of formula (I), or a pharmaceutically acceptable salt thereof, wherein at least one of the additional compounds is effective to inhibit the function of a target selected from HCV metalloprotease, HCV serine protease, HCV polymerase, HCV helicase, HCV NS4B portein, HCV entry, HCV assembly, HCV egress, HCV NS5A protein, and IMPDH for the treatment of an HCV infection.

Other embodiments of the present disclosure may comprise suitable combinations of two or more of embodiments and/or aspects disclosed herein.

Yet other embodiments and aspects of the disclosure will be apparent according to the description provided below.

The compounds of the present disclosure also exist as tautomers; therefore the present disclosure also encompasses all tautomeric forms.

The description of the present disclosure herein should be construed in congruity with the laws and principals of chemical bonding.

It should be understood that the compounds encompassed by the present disclosure are those that are suitably stable for use as pharmaceutical agent.

All patents, patent applications, and literature references cited in the specification are herein incorporated by reference in their entirety. In the case of inconsistencies, the present disclosure, including definitions, will prevail.

As used in the present specification, the following terms have the meanings indicated:

As used herein, the singular forms “a”, “an”, and “the” include plural reference unless the context clearly dictates otherwise.

Asymmetric centers exist in the compounds of the present disclosure. These centers are designated by the symbols “R” or “S”, depending on the configuration of substituents around the chiral carbon atom. It should be understood that the disclosure encompasses all stereochemical isomeric forms, or mixtures thereof, which possess the ability to inhibit NS5A. Individual stereoisomers of compounds can be prepared synthetically from commercially available starting materials which contain chiral centers or by preparation of mixtures of steroisomeric products followed by separation such as conversion to a mixture of diastereomers followed by separation or recrystallization, chromatographic techniques, or direct separation on chiral chromatographic columns. Starting compounds of particular stereochemistry are either commercially available or can be made and resolved by techniques known in the art.

Certain compounds of the present disclosure may also exist in different stable conformational forms which may be separable. Torsional asymmetry due to restricted rotation about an asymmetric single bond, for example because of steric hindrance or ring strain, may permit separation of different conformers. The present disclosure includes each conformational isomer of these compounds and mixtures thereof.

The term “compounds of the present disclosure”, and equivalent expressions, are meant to embrace compounds of Formula (I), and pharmaceutically acceptable enantiomers, diastereomers, and salts thereof. Similarly, references to intermediates are meant to embrace their salts where the context so permits.

The present disclosure is intended to include all isotopes of atoms occurring in the present compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed. Such compounds may have a variety of potential uses, for example as standards and reagents in determining biological activity. In the case of stable isotopes, such compounds may have the potential to favorably modify biological, pharmacological, or pharmacokinetic properties.

The compounds of the present disclosure can exist as pharmaceutically acceptable salts. The term “pharmaceutically acceptable salt,” as used herein, represents salts or zwitterionic forms of the compounds of the present disclosure which are water or oil-soluble or dispersible, which are, within the scope of sound medical judgment, suitable for use in contact with the tissues of patients without excessive toxicity, irritation, allergic response, or other problem or complication commensurate with a reasonable benefit/risk ratio, and are effective for their intended use. The salts can be prepared during the final isolation and purification of the compounds or separately by reacting a suitable nitrogen atom with a suitable acid. Representative acid addition salts include acetate, adipate, alginate, citrate, aspartate, benzoate, benzenesulfonate, bisulfate, butyrate, camphorate, camphorsulfonate; digluconate, dihydrobromide, diydrochloride, dihydroiodide, glycerophosphate, hemisulfate, heptanoate, hexanoate, formate, fumarate, hydrochloride, hydrobromide, hydroiodide, 2-hydroxyethanesulfonate, lactate, maleate, mesitylenesulfonate, methanesulfonate, naphthylenesulfonate, nicotinate, 2-naphthalenesulfonate, oxalate, palmoate, pectinate, persulfate, 3-phenylproprionate, picrate, pivalate, propionate, succinate, tartrate, trichloroacetate, trifluoroacetate, phosphate, glutamate, bicarbonate, para-toluenesulfonate, and undecanoate. Examples of acids which can be employed to form pharmaceutically acceptable addition salts include inorganic acids such as hydrochloric, hydrobromic, sulfuric, and phosphoric, and organic acids such as oxalic, maleic, succinic, and citric.

When it is possible that, for use in therapy, therapeutically effective amounts of a compound of formula (I), as well as pharmaceutically acceptable salts thereof, may be administered as the raw chemical, it is possible to present the active ingredient as a pharmaceutical composition. Accordingly, the disclosure further provides pharmaceutical compositions, which include therapeutically effective amounts of compounds of formula (I) or pharmaceutically acceptable salts thereof, and one or more pharmaceutically acceptable carriers, diluents, or excipients. The term “therapeutically effective amount,” as used herein, refers to the total amount of each active component that is sufficient to show a meaningful patient benefit, e.g., a reduction in viral load. When applied to an individual active ingredient, administered alone, the term refers to that ingredient alone. When applied to a combination, the term refers to combined amounts of the active ingredients that result in the therapeutic effect, whether administered in combination, serially, or simultaneously. The compounds of formula (I) and pharmaceutically acceptable salts thereof, are as described above. The carrier(s), diluent(s), or excipient(s) must be acceptable in the sense of being compatible with the other ingredients of the formulation and not deleterious to the recipient thereof. In accordance with another aspect of the present disclosure there is also provided a process for the preparation of a pharmaceutical formulation including admixing a compound of formula (I), or a pharmaceutically acceptable salt thereof, with one or more pharmaceutically acceptable carriers, diluents, or excipients. The term “pharmaceutically acceptable,” as used herein, refers to those compounds, materials, compositions, and/or dosage forms which are, within the scope of sound medical judgment, suitable for use in contact with the tissues of patients without excessive toxicity, irritation, allergic response, or other problem or complication commensurate with a reasonable benefit/risk ratio, and are effective for their intended use.

Pharmaceutical formulations may be presented in unit dose forms containing a predetermined amount of active ingredient per unit dose. Dosage levels of between about 0.01 and about 250 milligram per kilogram (“mg/kg”) body weight per day, preferably between about 0.05 and about 100 mg/kg body weight per day of the compounds of the present disclosure are typical in a monotherapy for the prevention and treatment of HCV mediated disease. Typically, the pharmaceutical compositions of this disclosure will be administered from about 1 to about 5 times per day or alternatively, as a continuous infusion. Such administration can be used as a chronic or acute therapy. The amount of active ingredient that may be combined with the carrier materials to produce a single dosage form will vary depending on the condition being treated, the severity of the condition, the time of administration, the route of administration, the rate of excretion of the compound employed, the duration of treatment, and the age, gender, weight, and condition of the patient. Preferred unit dosage formulations are those containing a daily dose or sub-dose, as herein above recited, or an appropriate fraction thereof, of an active ingredient. Treatment may be initiated with small dosages substantially less than the optimum dose of the compound. Thereafter, the dosage is increased by small increments until the optimum effect under the circumstances is reached. In general, the compound is most desirably administered at a concentration level that will generally afford antivirally effective results without causing any harmful or deleterious side effects.

When the compositions of this disclosure comprise a combination of a compound of the present disclosure and one or more additional therapeutic or prophylactic agent, both the compound and the additional agent are usually present at dosage levels of between about 10 to 150%, and more preferably between about 10 and 80% of the dosage normally administered in a monotherapy regimen.

Pharmaceutical formulations may be adapted for administration by any appropriate route, for example by the oral (including buccal or sublingual), rectal, nasal, topical (including buccal, sublingual, or transdermal), vaginal, or parenteral (including subcutaneous, intracutaneous, intramuscular, intra-articular, intrasynovial, intrasternal, intrathecal, intralesional, intravenous, or intradermal injections or infusions) route. Such formulations may be prepared by any method known in the art of pharmacy, for example by bringing into association the active ingredient with the carrier(s) or excipient(s). Oral administration or administration by injection are preferred.

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil emulsions.

For instance, for oral administration in the form of a tablet or capsule, the active drug component can be combined with an oral, non-toxic pharmaceutically acceptable inert carrier such as ethanol, glycerol, water, and the like. Powders are prepared by pulverizing the compound to a suitable fine size and mixing with a similarly comminuted pharmaceutical carrier such as an edible carbohydrate, as, for example, starch or mannitol. Flavoring, preservative, dispersing, and coloring agent can also be present.

Capsules are made by preparing a powder mixture, as described above, and filling formed gelatin sheaths. Glidants and lubricants such as colloidal silica, talc, magnesium stearate, calcium stearate, or solid polyethylene glycol can be added to the powder mixture before the filling operation. A disintegrating or solubilizing agent such as agar-agar, calcium carbonate, or sodium carbonate can also be added to improve the availability of the medicament when the capsule is ingested.

Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, and the like. Lubricants used in these dosage forms include sodium oleate, sodium chloride, and the like. Disintegrators include, without limitation, starch, methyl cellulose, agar, betonite, xanthan gum, and the like. Tablets are formulated, for example, by preparing a powder mixture, granulating or slugging, adding a lubricant and disintegrant, and pressing into tablets. A powder mixture is prepared by mixing the compound, suitable comminuted, with a diluent or base as described above, and optionally, with a binder such as carboxymethylcellulose, an aliginate, gelating, or polyvinyl pyrrolidone, a solution retardant such as paraffin, a resorption accelerator such as a quaternary salt and/or and absorption agent such as betonite, kaolin, or dicalcium phosphate. The powder mixture can be granulated by wetting with a binder such as syrup, starch paste, acadia mucilage, or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc, or mineral oil. The lubricated mixture is then compressed into tablets. The compounds of the present disclosure can also be combined with a free flowing inert carrier and compressed into tablets directly without going through the granulating or slugging steps. A clear or opaque protective coating consisting of a sealing coat of shellac, a coating of sugar or polymeric material, and a polish coating of wax can be provided. Dyestuffs can be added to these coatings to distinguish different unit dosages.

Oral fluids such as solution, syrups, and elixirs can be prepared in dosage unit form so that a given quantity contains a predetermined amount of the compound. Syrups can be prepared by dissolving the compound in a suitably flavored aqueous solution, while elixirs are prepared through the use of a non-toxic vehicle. Solubilizers and emulsifiers such as ethoxylated isostearyl alcohols and polyoxyethylene sorbitol ethers, preservatives, flavor additive such as peppermint oil or natural sweeteners, or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax, or the like.

The compounds of formula (I), and pharmaceutically acceptable salts thereof, can also be administered in the form of liposome delivery systems, such as small unilamellar vesicles, large unilamellar vesicles, and multilamellar vesicles. Liposomes can be formed from a variety of phopholipids, such as cholesterol, stearylamine, or phophatidylcholines.

The compounds of formula (I) and pharmaceutically acceptable salts thereof may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamidephenol, polyhydroxyethylaspartamidephenol, or polyethyleneoxidepolylysine substituted with palitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates, and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research 1986, 3(6), 318.

Pharmaceutical formulations adapted for topical administration may be formulated as ointments, creams, suspensions, lotions, powders, solutions, pastes, gels, sprays, aerosols, or oils.

Pharmaceutical formulations adapted for rectal administration may be presented as suppositories or as enemas.

Pharmaceutical formulations adapted for nasal administration wherein the carrier is a solid include a course powder having a particle size for example in the range 20 to 500 microns which is administered in the manner in which snuff is taken, i.e., by rapid inhalation through the nasal passage from a container of the powder held close up to the nose. Suitable formulations wherein the carrier is a liquid, for administration as a nasal spray or nasal drops, include aqueous or oil solutions of the active ingredient.

Pharmaceutical formulations adapted for administration by inhalation include fine particle dusts or mists, which may be generated by means of various types of metered, dose pressurized aerosols, nebulizers, or insufflators.

Pharmaceutical formulations adapted for vaginal administration may be presented as pessaries, tampons, creams, gels, pastes, foams, or spray formulations.

Pharmaceutical formulations adapted for parenteral administration include aqueous and non-aqueous sterile injection solutions which may contain anti-oxidants, buffers, bacteriostats, and soutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

It should be understood that in addition to the ingredients particularly mentioned above, the formulations may include other agents conventional in the art having regard to the type of formulation in question, for example those suitable for oral administration may include flavoring agents.

The term “patient” includes both human and other mammals.

The term “treating” refers to: (i) preventing a disease, disorder or condition from occurring in a patient that may be predisposed to the disease, disorder, and/or condition but has not yet been diagnosed as having it; (ii) inhibiting the disease, disorder, or condition, i.e., arresting its development; and (iii) relieving the disease, disorder, or condition, i.e., causing regression of the disease, disorder, and/or condition.

The compounds of the present disclosure can also be administered with a cyclosporin, for example, cyclosporin A or other analogs working through similar mechanism. Cyclosporin A has been shown to be active against HCV in clinical trials (Hepatology 2003, 38, 1282; Biochem. Biophys. Res. Commun. 2004, 313, 42; J. Gastroenterol. 2003, 38, 567).

Table 1 below lists some illustrative examples of compounds that can be administered with the compounds of this disclosure. The compounds of the disclosure can be administered with other anti-HCV active compounds in combination therapy, either jointly or separately, or by combining the compounds into a composition.

TABLE 1

| | | Type of Inhibitor or | |
|---|---|---|---|
| Brand Name | Physiological Class | Target | Source Company |
| NIM811 | | Cyclophilin Inhibitor | Novartis |
| Zadaxin | | Immuno-modulator | Sciclone |
| Suvus | | Methylene blue | Bioenvision |
| Actilon | | TLR9 agonist | Coley |
| (CPG10101) | | | |
| Batabulin (T67) | Anticancer | β-tubulin inhibitor | Tularik Inc., South |
| | | | San Francisco, CA |
| ISIS 14803 | Antiviral | antisense | ISIS |
| | | | Pharmaceuticals Inc, |
| | | | Carlsbad, CA/Elan |
| | | | Phamaceuticals Inc., |
| | | | New York, NY |
| Summetrel | Antiviral | antiviral | Endo |
| | | | Pharmaceuticals |
| | | | Holdings Inc., |
| | | | Chadds Ford, PA |
| GS-9132 (ACH- | Antiviral | HCV Inhibitor | Achillion/Gilead |
| 806) | | | |
| Pyrazolopyrimidine | Antiviral | HCV Inhibitors | Arrow Therapeutics |
| compounds and | | | Ltd. |
| salts | | | |
| From WO- | | | |
| 2005047288 | | | |
| 26 May 2005 | | | |
| Levovirin | Antiviral | IMPDH inhibitor | Ribapharm Inc., |
| | | | Costa Mesa, CA |
| Merimepodib | Antiviral | IMPDH inhibitor | Vertex |
| (VX-497) | | | Pharmaceuticals |
| | | | Inc., Cambridge, |
| | | | MA |
| XTL-6865 (XTL- | Antiviral | monoclonal antibody | XTL |
| 002) | | | Biopharmaceuticals |
| | | | Ltd., Rehovot, Isreal |
| Telaprevir | Antiviral | NS3 serine protease | Vertex |
| (VX-950, LY- | | inhibitor | Pharmaceuticals |
| 570310) | | | Inc., Cambridge, |
| | | | MA/Eli Lilly and |
| | | | Co. Inc., |
| | | | Indianapolis, IN |
| HCV-796 | Antiviral | NS5B Replicase | Wyeth/Viropharma |
| | | Inhibitor | |
| NM-283 | Antiviral | NS5B Replicase | Idenix/Novartis |
| | | Inhibitor | |
| GL-59728 | Antiviral | NS5B Replicase | Gene Labs/ |
| | | Inhibitor | Novartis |
| GL-60667 | Antiviral | NS5B Replicase | Gene Labs/ |
| | | Inhibitor | Novartis |
| 2′C MeA | Antiviral | NS5B Replicase | Gilead |
| | | Inhibitor | |
| PSI 6130 | Antiviral | NS5B Replicase | Roche |
| | | Inhibitor | |
| R1626 | Antiviral | NS5B Replicase | Roche |
| | | Inhibitor | |
| 2′C Methyl | Antiviral | NS5B Replicase | Merck |
| adenosine | | Inhibitor | |
| JTK-003 | Antiviral | RdRp inhibitor | Japan Tobacco Inc., |
| | | | Tokyo, Japan |
| Levovirin | Antiviral | ribavirin | ICN |
| | | | Pharmaceuticals, |
| | | | Costa Mesa, CA |
| Ribavirin | Antiviral | ribavirin | Schering-Plough |
| | | | Corporation, |
| | | | Kenilworth, NJ |
| Viramidine | Antiviral | Ribavirin Prodrug | Ribapharm Inc., |
| | | | Costa Mesa, CA |
| Heptazyme | Antiviral | ribozyme | Ribozyme |
| | | | Pharmaceuticals |
| | | | Inc., Boulder, CO |
| BILN-2061 | Antiviral | serine protease | Boehringer |
| | | inhibitor | Ingelheim Pharma |
| | | | KG, Ingelheim, |
| | | | Germany |
| SCH 503034 | Antiviral | serine protease | Schering Plough |
| | | inhibitor | |
| Zadazim | Immune modulator | Immune modulator | SciClone |
| | | | Pharmaceuticals |
| | | | Inc., San Mateo, CA |
| Ceplene | Immunomodulator | immune modulator | Maxim |
| | | | Pharmaceuticals |
| | | | Inc., San Diego, CA |
| CellCept | Immunosuppressant | HCV IgG immuno- | F. Hoffmann-La |
| | | suppressant | Roche LTD, Basel, |
| | | | Switzerland |
| Civacir | Immunosuppressant | HCV IgG immuno- | Nabi |
| | | suppressant | Biopharmaceuticals |
| | | | Inc., Boca Raton, FL |
| Albuferon - α | Interferon | albumin IFN-α2b | Human Genome |
| | | | Sciences Inc., |
| | | | Rockville, MD |
| Infergen A | Interferon | IFN | InterMune |
| | | alfacon-1 | Pharmaceuticals |
| | | | Inc., Brisbane, CA |
| Omega IFN | Interferon | IFN-ω | Intarcia Therapeutics |
| IFN-β and EMZ701 | Interferon | IFN-β and EMZ701 | Transition |
| | | | Therapeutics Inc., |
| | | | Ontario, Canada |
| Rebif | Interferon | IFN-β1a | Serono, Geneva, |
| | | | Switzerland |
| Roferon A | Interferon | IFN-α2a | F. Hoffmann-La |
| | | | Roche LTD, Basel, |
| | | | Switzerland |
| Intron A | Interferon | IFN-α2b | Schering-Plough |
| | | | Corporation, |
| | | | Kenilworth, NJ |
| Intron A and | Interferon | IFN-α2b/α1-thymosin | RegeneRx |
| Zadaxin | | | Biopharma. Inc., |
| | | | Bethesda, MD/ |
| | | | SciClone |
| | | | Pharmaceuticals Inc, |
| | | | San Mateo, CA |
| Rebetron | Interferon | IFN-α2b/ribavirin | Schering-Plough |
| | | | Corporation, |
| | | | Kenilworth, NJ |
| Actimmune | Interferon | INF-γ | InterMune Inc., |
| | | | Brisbane, CA |
| Interferon-β | Interferon | Interferon-β-1a | Serono |
| Multiferon | Interferon | Long lasting IFN | Viragen/ |
| | | | Valentis |
| Wellferon | Interferon | Lympho-blastoid IFN- | GlaxoSmithKline |
| | | αn1 | plc, Uxbridge, UK |
| Omniferon | Interferon | natural IFN-α | Viragen Inc., |
| | | | Plantation, FL |
| Pegasys | Interferon | PEGylated IFN-α2a | F. Hoffmann-La |
| | | | Roche LTD, Basel, |
| | | | Switzerland |
| Pegasys and | Interferon | PEGylated IFN-α2a/ | Maxim |
| Ceplene | | immune modulator | Pharmaceuticals |
| | | | Inc., San Diego, CA |
| Pegasys and | Interferon | PEGylated IFN- | F. Hoffmann-La |
| Ribavirin | | α2a/ribavirin | Roche LTD, Basel, |
| | | | Switzerland |
| PEG-Intron | Interferon | PEGylated IFN-α2b | Schering-Plough |
| | | | Corporation, |
| | | | Kenilworth, NJ |
| PEG-Intron/ | Interferon | PEGylated IFN- | Schering-Plough |
| Ribavirin | | α2b/ribavirin | Corporation, |
| | | | Kenilworth, NJ |
| IP-501 | Liver protection | antifibrotic | Indevus |
| | | | Pharmaceuticals |
| | | | Inc., Lexington, MA |
| IDN-6556 | Liver protection | caspase inhibitor | Idun |
| | | | Pharmaceuticals |
| | | | Inc., San Diego, CA |
| ITMN-191 (R-7227) | Antiviral | serine protease | InterMune |
| | | inhibitor | Pharmaceuticals |
| | | | Inc., Brisbane, CA |
| GL-59728 | Antiviral | NS5B Replicase | Genelabs |
| | | Inhibitor | |
| ANA-971 | Antiviral | TLR-7 agonist | Anadys |
| Boceprevir | Antiviral | serine protease | Schering Plough |
| | | inhibitor | |
| TMS-435 | Antiviral | serine protease | Tibotec BVBA, |
| | | inhibitor | Mechelen, Belgium |
| BI-201335 | Antiviral | serine protease | Boehringer |
| | | inhibitor | Ingelheim Pharma |
| | | | KG, Ingelheim, |
| | | | Germany |
| MK-7009 | Antiviral | serine protease | Merck |
| | | inhibitor | |
| PF-00868554 | Antiviral | replicase inhibitor | Pfizer |
| ANA598 | Antiviral | Non-Nucleoside | Anadys |
| | | NS5B Polymerase | Pharmaceuticals, |
| | | Inhibitor | Inc., San Diego, CA, |
| | | | USA |
| IDX375 | Antiviral | Non-Nucleoside | Idenix |
| | | Replicase Inhibitor | Pharmaceuticals, |
| | | | Cambridge, MA, |
| | | | USA |
| BILB 1941 | Antiviral | NS5B Polymerase | Boehringer |
| | | Inhibitor | Ingelheim Canada |
| | | | Ltd R&D, Laval, |
| | | | QC, Canada |
| PSI-7851 | Antiviral | Nucleoside | Pharmasset, |
| | | Polymerase Inhibitor | Princeton, NJ, USA |
| PSI-7977 | Antiviral | Nucleotide NS5B | Pharmasset, |
| | | Polymerase Inhibitor | Princeton, NJ, USA |
| VCH-759 | Antiviral | NS5B Polymerase | ViroChem Pharma |
| | | Inhibitor | |
| VCH-916 | Antiviral | NS5B Polymerase | ViroChem Pharma |
| | | Inhibitor | |
| GS-9190 | Antiviral | NS5B Polymerase | Gilead |
| | | Inhibitor | |
| Peg-interferon | Antiviral | Interferon | ZymoGenetics/Bristol- |
| lamda | | | Myers Squibb |
| daclatasvir | Antiviral | NS5A inhibitor | Bristol-Myers |
| | | | Squibb |
| BMS-791325 | Antiviral | NS5B Polymerase | Bristol-Myers |
| | | Inhibitor | Squibb |
| ACH-3102 | Antiviral | NS5A inhibitor | Bristol-Myers |
| | | | Squibb |
| asunaprevir | Antiviral | serine protease | Bristol-Myers |
| | | inhibitor | Squibb |
| IDX-719 | Antiviral | NS5A inhibitor | Idenix |
| GS-5885 | Antiviral | NS5A inhibitor | Gilead |
| GS-5816 | Antiviral | NS5A inhibitor | Gilead |
| ABT-267 | Antiviral | NS5A inhibitor | Abbvie |
| GSK-2336805 | Antiviral | NS5A inhibitor | GlaxoSmithKline |
| PPI-461 | Antiviral | NS5A inhibitor | Presidio |
| EDP-239 | Antiviral | NS5A inhibitor | Enanta |

The compounds of the present disclosure may also be used as laboratory reagents. Compounds may be instrumental in providing research tools for designing of viral replication assays, validation of animal assay systems and structural biology studies to further enhance knowledge of the HCV disease mechanisms. Further, the compounds of the present disclosure are useful in establishing or determining the binding site of other antiviral compounds, for example, by competitive inhibition.

The compounds of this disclosure may also be used to treat or prevent viral contamination of materials and therefore reduce the risk of viral infection of laboratory or medical personnel or patients who come in contact with such materials, e.g., blood, tissue, surgical instruments and garments, laboratory instruments and garments, and blood collection or transfusion apparatuses and materials.

This disclosure is intended to encompass compounds having formula (I) when prepared by synthetic processes or by metabolic processes including those occurring in the human or animal body (in vivo) or processes occurring in vitro.

The abbreviations used in the present application, including particularly in the examples which follow, are well-known to those skilled in the art. Some of the abbreviations used are as follows: h, hr, or hrs for hours; EtOAc for ethyl acetate; Hex for hexanes; DCM for dichloromethane; DEAD for diethyl azodicarboxylate; Ph₃P for triphenylphosphine; Et₂O for diethyl ether; THF for tetrahydrofuran; LiHMDS for lithium hexamethyldisilazide; Ph for phenyl; DIEA or DIPEA or iPr₂EtN for diiosopropylethylamine; EtOH for ethanol; MeOH for methanol; DMSO for dimethylsulfoxide; RT or Rt or rt or Rₜ for room temperature or retention time (context will dictate); ON or o/n for overnight; min for minutes; DCM for dichloromethane; HATU for O-(7-azabenzotriazol-1-yl)-N,N,N′,N′-tetramethyluronium hexafluorophosphate; DMF for N,N-dimethylformamide; TFA for trifluoroacetic acid; HOBt or HOBT for hydroxybenzotriazole; DME for 1,2-dimethoxyethane; and DMAP for N,N-dimethylaminopyridine.

### Int-6 Synthesis

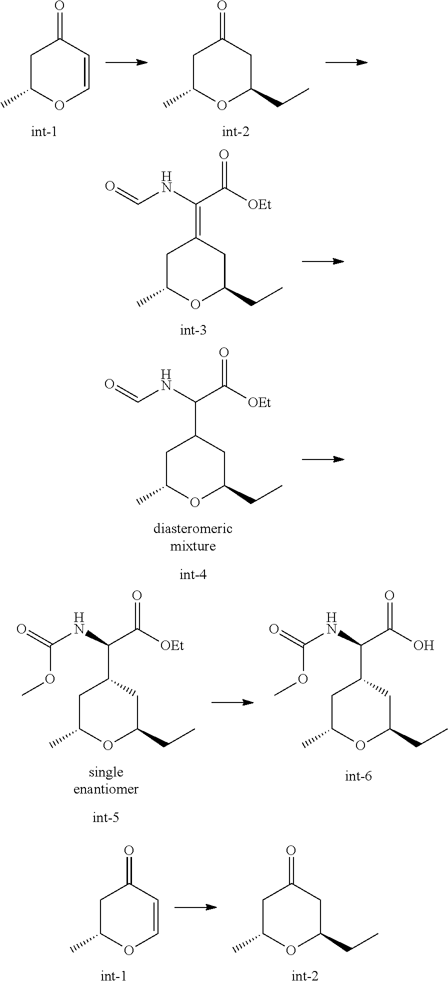

### Int-2

### (2R,6R)-2-ethyl-6-methyldihydro-2H-pyran-4(3H)-one

A solution of 0.5M ethyl lithium (407 mL, 203 mmol) in benzene/cyclohexane (9:1) was added (via a cannula) over 10 min to a stirred slurry of copper(I) iodide (20.7 g, 108 mmol) in Et₂O (100 mL) which was cooled to 0° C. The reaction solution was stirred at 0° C. for 1.5 h and then (R)-2-methyl-2H-pyran-4(3H)-one (7.6 g, 67.8 mmol) in Et₂O (25 mL) was added dropwise over 10 min. The reaction mixture was allowed to warm to RT and stirred 1.5 h. The reaction was poured into a stirred and cooled (0° C.) solution of sat. NH₄Cl (aq.) (˜800 mL) and water (˜200 mL). The biphasic solution was allowed to warm up to RT and stirred ON. The layers were separated and the organic component was dried (MgSO₄), filtered, concentrated and the crude material was purified using a Biotage Horizon (160 g SiO₂, 2% Et₂O in DCM, column was pre-equilibrated) to yield (2R,6R)-2-ethyl-6-methyldihydro-2H-pyran-4(3H)-one (2.23 g) as a yellow oil. ¹H NMR (400 MHz, CDCl₃) δ 4.26 (quin d, J=6.5, 4.8 Hz, 1H), 4.04 (dq, J=8.1, 5.7 Hz, 1H), 2.55 (dddd, J=14.2, 9.3, 4.8, 1.4 Hz, 2H), 2.26 (dtd, J=14.3, 6.5, 1.6 Hz, 2H), 1.67 (ddd, J=14.0, 8.1, 7.3 Hz, 1H), 1.56-1.44 (m, 1H), 1.28 (d, J=6.5 Hz, 3H), 0.96 (t, J=7.4 Hz, 3H).

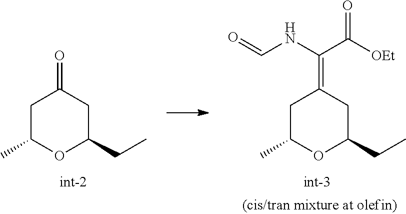

### Int-3

### ethyl 2-((2R,6R)-2-ethyl-6-methyldihydro-2H-pyran-4(3H)-ylidene)-2-formamidoacetate

Ethyl 2-isocyanoacetate (1.702 mL, 15.26 mmol) was added to a stirred suspension of cuprous oxide (0.109 g, 0.763 mmol) in Et₂O (50 mL) and the reaction mixture was stirred at RT for 10 min. Then (2R,6R)-2-ethyl-6-methyldihydro-2H-pyran-4(3H)-one (2.17 g, 15.2 mmol) in Et₂O (10 mL) was added and the reaction mixture was stirred at RT for 2.5 h. The reaction was then cooled to 0° C., treated with 1M KOtBu (15.3 mL, 15.3 mmol) in THF and stirred at 0° C. for 1 h. Then acetic acid (1.05 mL, 18.3 mmol) in DCM (15 mL) was added and the reaction was allowed to warm up to RT and stirred ON. The reaction was diluted with EtOAc (100 mL) and DCM (30 mL), washed with brine (100 mL) and the aqueous component was further extracted with EtOAc (50 mL). The combined organic component was dried (Na₂SO₄), filtered, concentrated and purified (110 g Thomson silica gel cartridge, gradient elution was performed from 15% to 100% B over 2 L where A=20% Et₂O/DCM, B=80% Et₂O/DCM) to yield ethyl 2-((2R,6R)-2-ethyl-6-methyldihydro-2H-pyran-4(3H)-ylidene)-2-formamidoacetate (3.65 g) as a mixture (˜1:1) of olefin isomers. LC/MS retention time 3.9 and 4.0 min (˜1:1); m/z 256.3 and 256.3 (MH+). LC data was recorded on a Shimadzu chromatograph equipped with a Phenomenex LUNA C18, 50×2 mm, 3 μm particles. The elution conditions employed a flow rate of 0.8 mL/min, a gradient of 100% solvent A/0% solvent B to 85% solvent A/15% solvent B, a gradient time of 4 min, a hold time of 1 min, and an analysis time of 5 min where solvent A was 10% CH₃CN/90% H₂O/0.1% TFA and solvent B was 10% H₂O/90% CH₃CN/0.1% TFA. MS data was determined using a Micromass Platform for LC in electrospray mode.

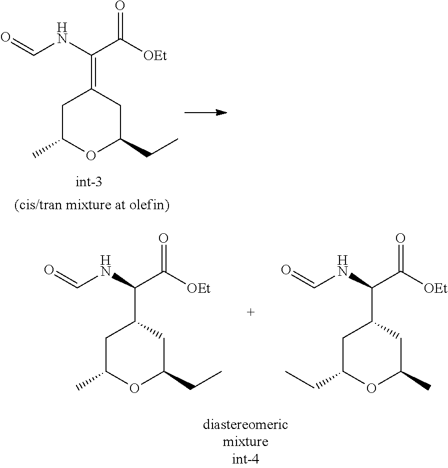 [SMILES: CCOC(=O)/C(NC=O)=C1\C[C@@H](CC)O[C@H](C)C1 ; CCOC(=O)[C@H](NC=O)[C@@H]1C[C@@H](CC)O[C@H](C)C1 ; CCOC(=O)[C@H](NC=O)[C@H]1C[C@@H](CC)O[C@H](C)C1]

### Int-4

### (S)-ethyl 2-((2R,4S,6R)-2-ethyl-6-methyltetrahydro-2H-pyran-4-yl)-2-formamidoacetate

### and

### (S)-ethyl 2-((2R,4R,6R)-2-ethyl-6-methyltetrahydro-2H-pyran-4-yl)-2-formamidoacetate

(−)-1,2-Bis((2,S,5S)-2,5-dimethylphospholano)ethane(cyclooctadiene)-rhodium(I)-tetrafluorborate (0.107 g, 0.193 mmol) was added to a solution of ethyl 2-((2R,6R)-2-ethyl-6-methyldihydro-2H-pyran-4(3H)-ylidene)-2-formamidoacetate (3.65 g, 14.3 mmol) (˜1:1 mixture of olefin isomers) in MeOH (100 mL) in a 500 mL Parr bottle under nitrogen. The reaction vessel was vacuum-flushed with nitrogen (3×) and then with hydrogen (3×) and shaken under 60 psi of hydrogen for three days. The reaction mixture was concentrated and the residual material purified (110 g Thomson silica gel cartridge, gradient elution was performed from 15% to 100% B/A over 2 L where A=20% Et₂O/DCM, B=80% Et₂O/DCM) to yield a diastereomeric mixture (2.426 g) of the title compounds. LC/MS retention time 1.9 min; m/z 258.3 (MH+). Diastereomers did not differentiate under these LC/MS conditions. LC data was recorded on a Shimadzu chromatograph equipped with a Phenomenex LUNA C18, 50×2 mm 3 μm. The elution conditions employed a flow rate of 0.8 mL/min, a gradient of 100% solvent A/0% solvent B to 0% solvent A/100% solvent B, a gradient time of 4 min, a hold time of 1 min, and an analysis time of 5 min where solvent A was 10% CH₃CN/90% H₂O/0.1% TFA and solvent B was 10% H₂O/90% CH₃CN/0.1% TFA. MS data was determined using a Micromass Platform for LC in electrospray mode.

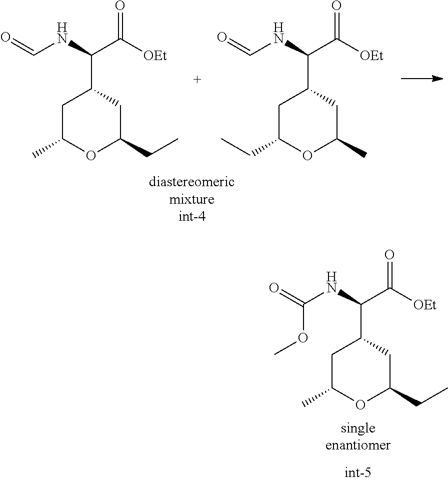

### Int-5

### (S)-ethyl 2-((2R,4S,6R)-2-ethyl-6-methyltetrahydro-2H-pyran-4-yl)-2-((methoxycarbonyl)amino)acetate

A solution of 1.5 N aq. HCl (15.5 mL, 23.3 mmol) was added to a solution of (S)-ethyl 2-((2R,4S,6R)-2-ethyl-6-methyltetrahydro-2H-pyran-4-yl)-2-formamidoacetate and (S)-ethyl 2-((2R,4R,6R)-2-ethyl-6-methyltetrahydro-2H-pyran-4-yl)-2-formamidoacetate (2.4 g, 9.33 mmol, ˜1:1) in EtOH (20 mL) and the reaction mixture was stirred at 52° C. for 16 h. The reaction was allowed to cool to RT, concentrated and then azeotroped with EtOH to afford a crude white solid. This crude material was dissolved in DCM (30 mL) and cooled in ice/water bath. Then methyl chloroformate (1.08 mL, 14.0 mmol) and DIPEA (4.89 mL, 28.0 mmol) were added and the mixture was stirred at RT for 16 h. The reaction was diluted with EtOAc (50 mL), washed with brine (30 mL) and the aqueous component was extracted with EtOAc (30 mL). The combined organic component was dried (Na₂SO₄), filtered, concentrated and purified by flash chromatography (80 g silica gel, loading solvent: DCM, elution: 0-30% EtOAc/hexanes) to yield a mixture of (S)-ethyl 2-((2R,4S,6R)-2-ethyl-6-methyltetrahydro-2H-pyran-4-yl)-2-((methoxycarbonyl) amino)acetate and (S)-ethyl 2-((2R,4R,6R)-2-ethyl-6-methyltetrahydro-2H-pyran-4-yl)-2-((methoxycarbonyl)amino)acetate (2.25 g) as clear colorless gel. The mixture of stereoisomers (2.1 g) was then separated by chiral super critical fluid chromatography:

### Preparative Column: Chiralpak AD-H (5×25 cm, 5 μm)

BPR pressure: 100 bars

### Temperature: 40° C.

Flow rate: 240 mL/min

### Mobile Phase: CO₂/EtOH (90/10)

### Detector Wavelength: 212 nm

Separation Program: Sequence injection
Injection: 0.83 mL with cycle time 3.3 min
Sample preparation: 2.1 g/50 mL EtOH, 42.0 mg/mL

(S)-ethyl 2-((2R,4S,6R)-2-ethyl-6-methyltetrahydro-2H-pyran-4-yl)-2-((methoxycarbonyl)amino)acetate (0.83 g) was retrieved as the second eluting peak. The relative stereochemistry was assigned based on NOE analysis of this product and the other diastereomer (first eluting peak) isolated. LC/MS retention time 3.98 min; m/z 310.14 (MNa+). LC data was recorded on a Shimadzu chromatograph equipped with a Phenomenex LUNA C18, 50×2 mm, 3 μm particles. The elution conditions employed a flow rate of 0.8 mL/min, a gradient of 100% solvent A/0% solvent B to 0% solvent A/100% solvent B, a gradient time of 4 min, a hold time of 1 min, and an analysis time of 5 min where solvent A was 10% MeOH/90% H₂O/0.1% TFA and solvent B was 10% H₂O/90% MeOH/0.1% TFA. MS data was determined using a Micromass Platform for LC in electrospray mode. ¹H NMR (400 MHz, CDCl₃) δ 5.23 (d, J=8.8 Hz, 1H), 4.34-4.28 (m, 1H), 4.23 (tdd, J=13.9, 7.2, 3.8 Hz, 2H), 3.92-3.83 (m, 1H), 3.70 (s, 3H), 3.69-3.61 (m, 1H), 2.31-2.19 (m, 1H), 1.89-1.75 (m, 1H), 1.62-1.48 (m, 2H), 1.38 (td, J=13.9, 6.8 Hz, 2H), 1.30 (t, J=7.2 Hz, 3H), 1.14 (d, J=6.0 Hz, 3H), 1.12-1.02 (m, 1H), 0.90 (t, J=7.4 Hz, 3H).

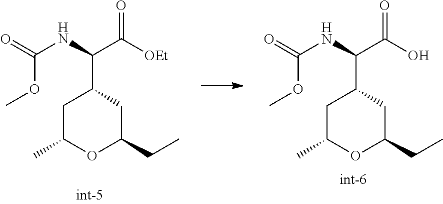 [SMILES: CCOC(=O)[C@H](NC(=O)OC)[C@@H]1C[C@@H](CC)O[C@H](C)C1 ; CC[C@@H]1C[C@@H]([C@@H](NC(=O)OC)C(=O)O)C[C@@H](C)O1]

### Int-6

### (S)-2-((2R,4S,6R)-2-ethyl-6-methyltetrahydro-2H-pyran-4-yl)-2-((methoxycarbonyl)amino)acetic acid

A solution of 1M LiOH (4.2 mL, 4.2 mmol) was added to a solution of (2S)-ethyl 2-((2R,6R)-2-ethyl-6-methyltetrahydro-2H-pyran-4-yl)-2-((methoxycarbonyl)amino)acetate (0.58 g, 2.018 mmol) in THF (10 mL) and the reaction mixture was stirred at RT for 16 h. The reaction was neutralized with aq. 1M HCl (4.2 mL) and extracted with EtOAc (50 mL and 20 mL). The combined organic component was dried (Na₂SO₄), filtered and concentrated to yield (2S)-2-((2R,6R)-2-ethyl-6-methyltetrahydro-2H-pyran-4-yl)-2-((methoxycarbonyl)amino)acetic acid (0.53 g) as white solid. LC/MS retention time 3.6 min; m/z 282.21 (MNa+). LC data was recorded on a Shimadzu chromatograph equipped with a Phenomenex LUNA C18, 50×2 mm, 3 um. The elution conditions employed a flow rate of 0.8 mL/min, a gradient of 100% solvent A/0% solvent B to 0% solvent A/100% solvent B, a gradient time of 4 min, a hold time of 1 min, and an analysis time of 5 min where solvent A was 10% MeOH/90% H₂O/0.1% TFA and solvent B was 10% H₂O/90% MeOH/0.1% TFA. MS data was determined using a Micromass Platform for LC in electrospray mode. ¹H NMR (400 MHz, CDCl₃) (˜5:1 mixture of rotamers, only major reported) δ 6.03 (br s, 1H), 5.30 (d, J=8.8 Hz, 1H), 4.35 (dd, J=8.7, 4.9 Hz, 1H), 3.97-3.88 (m, 1H), 3.79-3.64 (m, 4H), 2.39-2.25 (m, 1H), 1.93-1.76 (m, 1H), 1.69-1.54 (m, 2H), 1.49-1.35 (m, 2H), 1.16 (d, J=6.0 Hz, 3H), 1.14-1.05 (m, 1H), 0.91 (t, J=7.4 Hz, 3H).

### Int-13 Synthesis

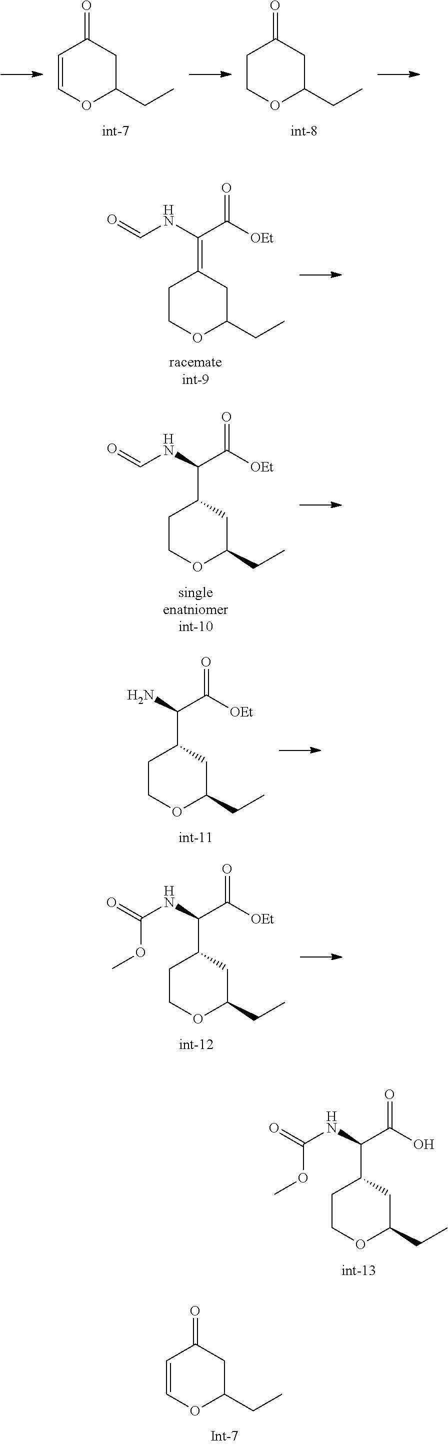

### Int-7

### 2-ethyl-2H-pyran-4(3H)-one

(Ref: Danishefsky, S.; Kerwin, Jr J. F. J. Org. Chem, 1982, 47, 1597) Propionaldehyde (3.60 mL, 48.0 mmol) was added to a stirred solution of (E)-((4-methoxybuta-1,3-dien-2-yl)oxy)trimethylsilane (4.4 g, 24 mmol) in Et₂O (100 mL) at −78° C. under nitrogen. Then (diethyloxonio)trifluoroborate (3.08 mL, 24.5 mmol) was added dropwise over ˜10 min. and the reaction mixture was stirred at −78° C. for 3 h. The reaction was quenched with sat. NaHCO₃ (aq) (40 mL), allowed to warm up to RT and stirred ON. The layers were separated and the aqueous component was extracted with Et₂O (2×100 mL). The combined organic component was dried (Na₂SO₄), filtered and concentrated to a yellow oil which was then purified using a Biotage Horizon (90 g SiO₂, 20% EtOAc/hexanes) to give 2-ethyl-2H-pyran-4(3H)-one (2.98 g) as a clear yellow oil.

¹H NMR (400 MHz, CDCl₃-d) δ 7.38 (d, J=6.0 Hz, 1H), 5.41 (dd, J=6.0, 1.3 Hz, 1H), 4.41-4.30 (m, 1H), 2.61-2.38 (m, 2H), 1.92-1.67 (m, 2H), 1.04 (t, J=7.5 Hz, 3H)

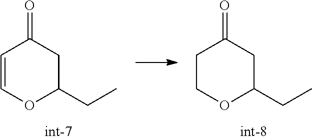

### Int-8

### 2-ethyldihydro-2H-pyran-4(3H)-one

10% Pd/C (0.706 g, 0.663 mmol) was added to a solution of 2-ethyl-2H-pyran-4(3H)-one (2.79 g, 22.12 mmol) in EtOAc (50 mL). The reaction vessel was sealed, vacuum flushed with nitrogen (4×) and with hydrogen (4×) and then shaken on a Parr shaker under 20 psi of hydrogen at RT for 16 h. The reaction mixture was filtered, concentrated and purified by flash silica chromatography (loading solvent: Et₂O, eluted with 20% EtOAc/hexanes) to yield 2-ethyldihydro-2H-pyran-4(3H)-one (1.45 g) as clear colorless oil. ¹H NMR (400 MHz, CDCl₃) δ 4.23 (ddd, J=11.4, 7.4, 1.5 Hz, 1H), 3.60 (ddd, J=12.3, 11.4, 2.9 Hz, 1H), 3.50-3.39 (m, 1H), 2.59-2.46 (m, 1H), 2.38-2.30 (m, 1H), 2.29-2.17 (m, 2H), 1.61 (dq, J=14.2, 7.2 Hz, 1H), 1.56-1.44 (m, 1H), 0.91 (t, J=7.5 Hz, 3H).

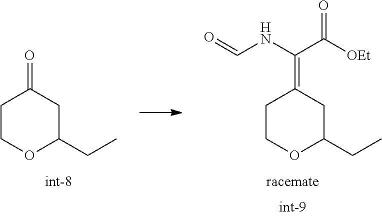

### Int-9

### (E)-ethyl 2-(2-ethyldihydro-2H-pyran-4(3H)-ylidene)-2-formamidoacetate

Cuprous oxide (0.066 g, 0.46 mmol) was added to a solution of ethyl 2-isocyanoacetate (1.12 mL, 10.2 mmol) in Et₂O (20 mL) and the mixture was stirred at RT for 10 min. Then 2-ethyldihydro-2H-pyran-4(3H)-one (1.19 g, 9.28 mmol) in Et₂O (10 mL) was added, and the reaction mixture was stirred at RT for 3 h. The reaction was cooled to 0° C. and treated with 1M KOtBu (11.14 mL, 11.14 mmol) in THF. The reaction was stirred at 0° C. for 30 min and then acetic acid (0.691 mL, 12.1 mmol) in DCM (10 mL) was added and the reaction was allowed to warm up to RT and stirred ON. The reaction was diluted with EtOAc (100 mL), partitioned with brine (50 mL) and the aqueous component was extracted with EtOAc (50 mL). The combined organic component was dried (Na₂SO₄), filtered and concentrated. The crude material was purified by flash silica chromatography (loading solvent: DCM, eluted with 50% EtOAc/hexanes) to independently yield two stereoisomeric products, each as an enantiomeric pair. The second eluting peak was determined to be racemic (E)-ethyl 2-(2-ethyldihydro-2H-pyran-4(3H)-ylidene)-2-formamidoacetate (858 mg) by ¹H NMR NOE analysis and was isolated as white solid. LC/MS retention time 2.92 min; m/z 264.13 (MNa+). LC data was recorded on a Shimadzu chromatograph equipped with a Phenomenex LUNA C18, 50×2 mm, 3 μm particles. The elution conditions employed a flow rate of 0.8 mL/min, a gradient of 100% solvent A/0% solvent B to 0% solvent A/100% solvent B, a gradient time of 4 min, a hold time of 1 min, and an analysis time of 5 min where solvent A was 10% MeOH/90% H₂O/0.1% TFA and solvent B was 10% H₂O/90% MeOH/0.1% TFA. MS data was determined using a Micromass Platform for LC in electrospray mode. ¹H NMR (400 MHz, CDCl₃) (7:3 mixture of rotamers) δ 8.24 (d, J=1.5 Hz, 0.7H), 7.96 (d, J=11.5 Hz, 0.3H), 6.75 (br. s., 0.7H), 6.60 (d, J=11.8 Hz, 0.3H), 4.31-4.21 (m, 2H), 4.19-4.07 (m, 1H), 3.75-3.62 (m, 1H), 3.55 (td, J=11.4, 2.8 Hz, 0.7H), 3.44 (td, J=11.7, 2.5 Hz, 0.3H), 3.38-3.25 (m, 1H), 2.73-2.65 (m, 0.3H), 2.45-2.37 (m, 0.7H), 2.36-2.22 (m, 1H), 2.04 (dd, J=13.9, 10.9 Hz, 1H), 1.71-1.50 (m, 2H), 1.36-1.29 (m, 3H), 1.02-0.95 (m, 3H).

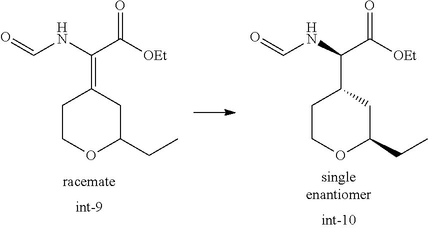 [SMILES: CCOC(=O)/C(NC=O)=C1/CCOC(CC)C1 ; CCOC(=O)[C@H](NC=O)[C@H]1CCO[C@H](CC)C1]

### Int-10

### (S)-ethyl 2-((2R,4S)-2-ethyltetrahydro-2H-pyran-4-yl)-2-formamidoacetate

In a Parr shaker vessel (−)-1,2-bis((2S,5S)-2,5-dimethylphospholano)ethane(cyclooctadiene)-rhodium (I) tetrafluoroborate (135 mg, 0.243 mmol) was added to a solution of racemic (E)-ethyl 2-(2-ethyldihydro-2H-pyran-4(3H)-ylidene)-2-formamidoacetate (840 mg, 3.48 mmol) in MeOH (40 mL). The reaction vessel was vacuum flushed with nitrogen (4×) and then with hydrogen (4×) and shaken under hydrogen (55 psi) at RT for 3 days. The reaction was concentrated and then purified by flash silica chromatography (loading solvent: DCM, eluted with 50% EtOAc/hexanes) to yield a diastereomeric mixture (650 mg) of (S)-ethyl 2-((2R,4S)-2-ethyltetrahydro-2H-pyran-4-yl)-2-formamidoacetate and (S)-ethyl 2-((2S,4S)-2-ethyltetrahydro-2H-pyran-4-yl)-2-formamidoacetate as a clear colorless gel. The diastereomeric mixture was separated by preparative HPLC (C18, H₂O/CH₃CN with 10 mM NH₄OAc buffer) to independently isolate each stereoisomer. The first eluting peak was determined to be (S)-ethyl 2-((2R,4S)-2-ethyltetrahydro-2H-pyran-4-yl)-2-formamidoacetate (312 mg) by ¹H NMR analysis of the coupling constants (pyrane ring determined to be in a boat conformation and thus it was assigned as the trans isomer) and was isolated as a clear colorless gel. The second eluting peak was determined to be (S)-ethyl 2-((2S,4S)-2-ethyltetrahydro-2H-pyran-4-yl)-2-formamidoacetate (181 mg) by ¹H NMR analysis of the coupling constants (pyrane ring determined to be in a chair conformation and thus it was assigned as the cis isomer) and was isolated as a clear colorless gel.

Data for (S)-ethyl 2-((2R,4S)-2-ethyltetrahydro-2H-pyran-4-yl)-2-formamidoacetate: LC/MS retention time 2.70 min; m/z 266.16 (MNa+). LC data was recorded on a Shimadzu chromatograph equipped with a Phenomenex LUNA C18, 50×2 mm, 3 μm particles. The elution conditions employed a flow rate of 0.8 mL/min, a gradient of 100% solvent A/0% solvent B to 0% solvent A/100% solvent B, a gradient time of 4 min, a hold time of 1 min, and an analysis time of 5 min where solvent A was 10% MeOH/90% H₂O/0.1% TFA and solvent B was 10% H₂O/90% MeOH/0.1% TFA. MS data was determined using a Micromass Platform for LC in electrospray mode. ¹H NMR (400 MHz, CDCl₃) (10:1 mixture of rotamers, only major reported) δ 8.26 (d, J=0.5 Hz, 1H), 6.17 (d, J=8.8 Hz, 1H), 4.89 (t, J=8.7 Hz, 1H), 4.29-4.17 (m, 2H), 3.71 (t, J=5.5 Hz, 2H), 3.75-3.64 (m, 1H), 2.23-2.12 (m, 1H), 1.79-1.57 (m, 3H), 1.50-1.35 (m, 3H), 1.30 (t, J=7.2 Hz, 3H), 0.91 (t, J=7.4 Hz, 3H). Data for (S)-ethyl 2-((2S,4S)-2-ethyltetrahydro-2H-pyran-4-yl)-2-formamidoacetate: LC/MS retention time 2.79 min; m/z 266.16 (MNa+). LC data was recorded on a Shimadzu chromatograph equipped with a Phenomenex LUNA C18, 50×2 mm, 3 μm particles. The elution conditions employed a flow rate of 0.8 mL/min, a gradient of 100% solvent A/0% solvent B to 0% solvent A/100% solvent B, a gradient time of 4 min, a hold time of 1 min, and an analysis time of 5 min where solvent A was 10% MeOH/90% H₂O/0.1% TFA and solvent B was 10% H₂O/90% MeOH/0.1% TFA. MS data was determined using a Micromass Platform for LC in electrospray mode. ¹H NMR (400 MHz, CDCl₃) (10:1 mixture of rotamers, only major reported) δ 8.26 (s, 1H), 6.23 (d, J=8.5 Hz, 1H), 4.69 (dd, J=8.9, 4.9 Hz, 1H), 4.24 (q, J=7.0 Hz, 2H), 4.07-3.96 (m, 1H), 3.40 (td, J=11.9, 2.3 Hz, 1H), 3.23-3.10 (m, 1H), 2.19-2.04 (m, 1H), 1.61-1.35 (m, 5H), 1.31 (t, J=7.2 Hz, 3H), 1.16-1.04 (m, 1H), 0.92 (t, J=7.5 Hz, 3H).

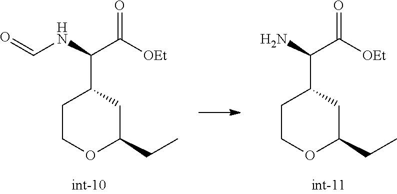 [SMILES: CCOC(=O)[C@H](NC=O)[C@H]1CCO[C@H](CC)C1 ; CCOC(=O)[C@H](N)[C@H]1CCO[C@H](CC)C1]

### Int-11

### (S)-ethyl 2-amino-2-((2R,4S)-2-ethyltetrahydro-2H-pyran-4-yl)acetate

A solution of 1.5 N aq. HCl (3.0 mL, 4.5 mmol) was added to a solution of (S)-ethyl 2-((2R,4S)-2-ethyltetrahydro-2H-pyran-4-yl)-2-formamidoacetate (242 mg, 0.995 mmol) in EtOH (3 mL) and the mixture was stirred at 52° C. for 16 h. The reaction mixture was shown to contain (S)-ethyl 2-amino-2-((2R,4S)-2-ethyltetrahydro-2H-pyran-4-yl)acetate and was used without further purification. LC/MS retention time 2.20 min; m/z 216.14 (MH+). LC data was recorded on a Shimadzu chromatograph equipped with a Phenomenex LUNA C18, 50×2 mm, 3 μm particles. The elution conditions employed a flow rate of 0.8 mL/min, a gradient of 100% solvent A/0% solvent B to 0% solvent A/100% solvent B, a gradient time of 4 min, a hold time of 1 min, and an analysis time of 5 min where solvent A was 10% MeOH/90% H₂O/0.1% TFA and solvent B was 10% H₂O/90% MeOH/0.1% TFA. MS data was determined using a Micromass Platform for LC in electrospray mode.

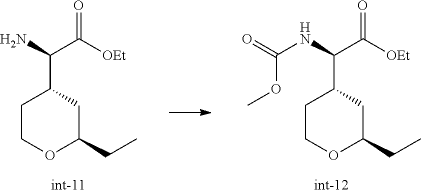 [SMILES: CCOC(=O)[C@H](N)[C@H]1CCO[C@H](CC)C1 ; CCOC(=O)[C@H](NC(=O)OC)[C@H]1CCO[C@H](CC)C1]

### Int-12

### (S)-ethyl 2-((2R,4S)-2-ethyltetrahydro-2H-pyran-4-yl)-2-((methoxycarbonyl)amino)acetate

Methyl chloroformate (0.231 mL, 2.98 mmol) and then DIPEA (1.910 mL, 10.93 mmol) were added to the stirred crude reaction mixture (crude Int-11) containing (S)-ethyl 2-amino-2-((2R,4S)-2-ethyltetrahydro-2H-pyran-4-yl)acetate (214 mg, 0.994 mmol) at 0° C. The reaction mixture was allowed to warm up to RT and then stirred for 5 h. The crude reaction was concentrated and the residue was diluted with brine and extracted with EtOAc (10 mL+5 mL). The combined organic component was dried, filtered, concentrated and purified by flash silica chromatography (loading solvent: DCM, eluted with 20% EtOAc/hexanes) to yield product (S)-ethyl 2-((2R,4S)-2-ethyltetrahydro-2H-pyran-4-yl)-2-((methoxycarbonyl)amino)acetate (181.6 mg) as a clear colorless gel. LC/MS retention time 2.95 min; m/z 296.18 (MNa+). LC data was recorded on a Shimadzu chromatograph equipped with a Phenomenex LUNA C18, 50×2 mm, 3 μm particles. The elution conditions employed a flow rate of 0.8 mL/min, a gradient of 100% solvent A/0% solvent B to 0% solvent A/100% solvent B, a gradient time of 4 min, a hold time of 1 min, and an analysis time of 5 min where solvent A was 10% MeOH/90% H₂O/0.1% TFA and solvent B was 10% H₂O/90% MeOH/0.1% TFA. MS data was determined using a Micromass Platform for LC in electrospray mode.

¹H NMR (400 MHz, CDCl₃) δ 5.16 (d, J=8.5 Hz, 1H), 4.50 (t, J=8.7 Hz, 1H), 4.22 (dtt, J=10.8, 7.3, 3.6 Hz, 2H), 3.78-3.61 (m, 3H), 3.70 (s, 3H), 2.20-2.07 (m, 1H), 1.76-1.61 (m, 3H), 1.51-1.36 (m, 3H), 1.30 (t, J=7.2 Hz, 3H), 0.92 (t, J=7.5 Hz, 3H).

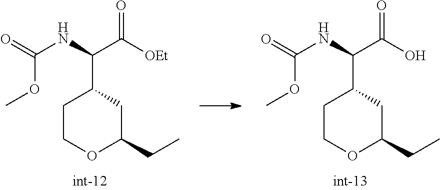 [SMILES: CCOC(=O)[C@H](NC(=O)OC)[C@H]1CCO[C@H](CC)C1 ; CC[C@@H]1C[C@@H]([C@@H](NC(=O)OC)C(=O)O)CCO1]

### Int-13

### (S)-2-((2R,4S)-2-ethyltetrahydro-2H-pyran-4-yl)-2-((methoxycarbonyl)amino)acetic acid

An aqueous 1M LiOH (1.19 mL, 1.19 mmol) solution was added to a solution of (S)-ethyl 2-((2R,4S)-2-ethyltetrahydro-2H-pyran-4-yl)-2-((methoxycarbonyl)amino)acetate (163 mg, 0.596 mmol) in THF (4 mL) and the reaction mixture was stirred at RT for 16 h. The reaction was neutralized with 1M HCl (1.2 mL) and extracted with EtOAc (10 mL+5 mL). The combined organic component was dried, filtered and concentrated to yield (S)-2-((2R,4S)-2-ethyltetrahydro-2H-pyran-4-yl)-2-((methoxycarbonyl)amino)acetic acid (151.8 mg) as clear colorless gum. LC/MS retention time 2.72 min; m/z 268.11 (MNa+). LC data was recorded on a Shimadzu chromatograph equipped with a Phenomenex LUNA C18, 50×2 mm, 3 μm particles. The elution conditions employed a flow rate of 0.8 mL/min, a gradient of 100% solvent A/0% solvent B to 0% solvent A/100% solvent B, a gradient time of 4 min, a hold time of 1 min, and an analysis time of 5 min where solvent A was 10% MeOH/90% H₂O/0.1% TFA and solvent B was 10% H₂O/90% MeOH/0.1% TFA. MS data was determined using a Micromass Platform for LC in electrospray mode.

### Alternative Synthesis:

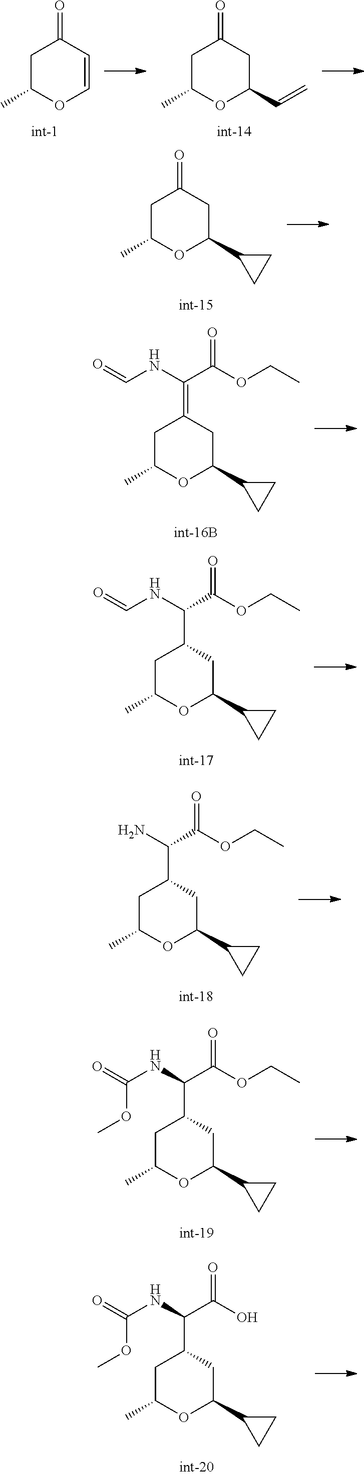

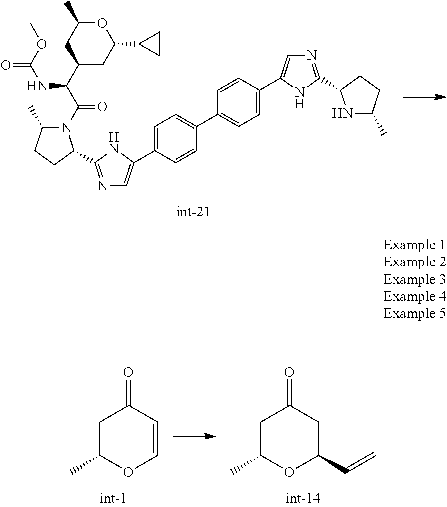

### Int-14

### (2R,6S)-2-methyl-6-vinyldihydro-2H-pyran-4(3H)-one

In a 1 L round bottomed flask copper(I)bromide-dimethyl sulfide complex (20.5 g, 100 mmol) was heated with a heat gun and then allowed to cool under high vacuum while stirring (3×). The stirring magnet was removed from the homogeneous solid and an overhead stirrer was affixed to the flask. The solids were slurried into THF (170 mL), the reaction mixture was cooled to −78° C. and 1M vinylmagnesium bromide (200 mL, 200 mmol) in THF was added dropwise over ˜50 min. The amber free flowing slurry was stirred at −78° C. for an additional 1 h, and then (R)-2-methyl-2H-pyran-4(3H)-one (14.0 g, 49.9 mmol) (˜60% w/w THF) in THF (30 mL) was added dropwise over 20 min. The reaction mixture was stirred at −78° C. for 2 h and then ½ sat. aq. NH₄Cl (200 mL) and Et₂O (˜200 mL) were added and the reaction was allowed to warm to RT and stirred ON. The crude biphasic emulsion was filtered through celite, the layers were separated and the aqueous component was further extracted with Et₂O (2×200 mL). The combined organic component was washed with brine (200 mL), dried (MgSO₄), filtered and concentrated. The crude material was purified on a Biotage Horizon (120 g SiO₂, 10-30% Et₂O/hexanes) to yield (2R,6S)-2-methyl-6-vinyldihydro-2H-pyran-4(3H)-one (3.9 g) as a yellow oil. ¹H NMR (400 MHz, CDCl₃) δ 5.92 (ddd, J=17.4, 10.8, 4.6 Hz, 1H), 5.33-5.21 (m, 2H), 4.78-4.72 (m, 1H), 4.26-4.16 (m, 1H), 2.66 (ddd, J=174.6, 6.0, 1.0 Hz, 1H), 2.54 (ddd, J=14.6, 4.5, 1.5 Hz, 1H), 2.47 (ddd, J=14.3, 3.8, 1.5 Hz, 1H), 2.26 (ddd, J=14.3, 8.5, 1.0 Hz, 1H), 1.29 (d, J=6.3 Hz, 3H).

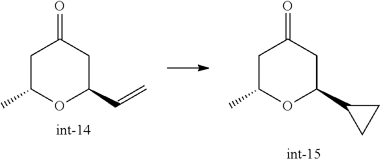

### Int-15

### (2S,6R)-2-cyclopropyl-6-methyldihydro-2H-pyran-4(3H)-one

(2R,6S)-2-Methyl-6-vinyldihydro-2H-pyran-4(3H)-one (3.60 g, 25.7 mmol) and DCM (125 mL) were added to a flask equipped with a graduated addition funnel, condenser with nitrogen inlet and a temperature probe for internal temperature monitoring. The clear solution was cooled in ice bath (0° C.) and then 1M diethylzinc (77 mL, 77 mmol) in hexanes was added dropwise via the addition funnel over 15 min. The reaction mixture was stirred for 5 min and then diiodomethane (20.7 mL, 257 mmol) was added via a cannulla over 25 min. NOTE: a controlled exothermic reaction was noted by a rise in temperature to ˜9° C. near the end of the addition. The reaction mixture was stirred in the ice bath for 15 min. The ice bath was removed, the reaction mixture was allowed to warm to RT and stirring was continued for 4 h. The reaction was then cooled to 0° C., quenched with sat. aq NH₄Cl (200 mL) and stirred ON. The reaction mixture was diluted with CHCl₃ (150 mL), the layers were separated and the aqueous component was further extracted with Et₂O (150 mL). The combined organic components were dried (MgSO₄), filtered, concentrated and the residue was purified by flash silica chromatography (240 g silica, 0-30% Et₂O/hexanes) to yield (2S,6R)-2-cyclopropyl-6-methyldihydro-2H-pyran-4(3H)-one (2.78 g) as clear colorless oil. ¹H NMR (400 MHz, CDCl₃) δ 4.46-4.36 (m, 1H), 3.36 (dt, J=8.9, 5.5 Hz, 1H), 2.63 (ddd, J=14.1, 5.3, 1.3 Hz, 1H), 2.56 (ddd, J=14.1, 4.5, 1.3 Hz, 1H), 2.45 (ddd, J=14.1, 5.8, 1.5 Hz, 1H), 2.26 (ddd, J=14.1, 7.3, 1.1 Hz, 1H), 1.28 (d, J=6.5 Hz, 3H), 1.06-0.95 (m, 1H), 0.65-0.55 (m, 2H), 0.44-0.38 (m, 1H), 0.25-0.19 (m, 1H).

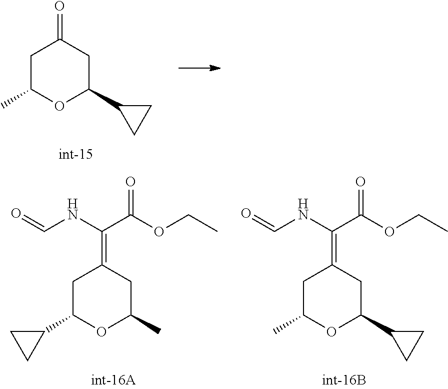

### Int-16

Ethyl 2-isocyanoacetate (0.84 mL, 7.5 mmol) was added to a suspension of cuprous oxide (0.054 g, 0.38 mmol) in Et₂O (20 mL) and the reaction mixture was stirred at RT for 10 min. Then (2S,6R)-2-cyclopropyl-6-methyldihydro-2H-pyran-4(3H)-one (1.16 g, 7.52 mmol) in Et₂O (10 mL) was added and the reaction mixture was stirred at RT for 3 h. The reaction mixture was cooled to 0° C. and then treated with 1M KOtBu (7.52 mL, 7.52 mmol) in THF. The reaction mixture was stirred 1 h at 0° C. and then acetic acid (0.560 mL, 9.78 mmol) in DCM (10 mL) was added and the reaction was allowed to warm up to RT and stirred ON. The reaction mixture was diluted with EtOAc (60 mL), partitioned with brine (50 mL) and the aqueous component was further extracted with EtOAc (30 mL). The combined organic component was dried (Na₂SO₄), filtered and concentrated. The crude amber oil was purified and the regioisomers seperated using a Biotage Horizon (80 g SiO₂, 0-50% EtOAc/hex) and then the mixed fractions were repurified by a second column (25 g SiO₂, 0-50% EtOAc/hex) to yield (Z)-ethyl 2-((2S,6R)-2-cyclopropyl-6-methyldihydro-2H-pyran-4(3H)-ylidene)-2-formamidoacetate (0.77 g) (the first eluting product from silica column) as white solid and (E)-ethyl 2-((2S,6R)-2-cyclopropyl-6-methyldihydro-2H-pyran-4(3H)-ylidene)-2-formamidoacetate (1.1 g, 4.11 mmol, 54.7% yield) (the second eluting product from silica column) also as white solid. The double bond geometry was determined by NOE analysis of each olefin isomer.

### Int-16A

### (Z)-ethyl 2-((2S,6R)-2-cyclopropyl-6-methyldihydro-2H-pyran-4(3H)-ylidene)-2-formamidoacetate

LC/MS retention time 1.20 min; m/z 268.2 (MH+). LC data was recorded on a Shimadzu chromatograph equipped with a Waters Aquity BEH C18 2.1×50 mm, 1.7 μm particles. The elution conditions employed a flow rate of 0.8 mL/min, a gradient of 70% solvent A/30% solvent B to 50% solvent A/50% solvent B, a gradient time of 1.5 min, a hold time of 0.5 min, and an analysis time of 2 min where solvent A was 10% MeOH/90% H₂O/0.1% TFA and solvent B was 10% H₂O/90% MeOH/0.1% TFA. MS data was determined using a Micromass Platform for LC in electrospray mode. ¹H NMR (400 MHz, CDCl₃) δ 8.25 (d, J=1.3 Hz, 0.7H), 7.99 (d, J=11.5 Hz, 0.3H), 6.84 (br. s., 0.7H), 6.73 (d, J=11.0 Hz, 0.3H), 4.29-4.15 (m, 3H), 3.20 (dd, J=14.3, 3.8 Hz, 0.3H), 3.11-3.02 (m, 1H), 2.99 (dd, J=14.2, 4.1 Hz, 0.7H), 2.82 (dd, J=14.1, 6.3 Hz, 0.7H), 2.70-2.61 (m, 0.6H), 2.57-2.48 (m, 1H), 2.32 (dd, J=13.9, 7.4 Hz, 0.7H), 1.35-1.28 (m, 3H), 1.26-1.20 (m, 3H), 1.04-0.94 (m, 1H), 0.61-0.46 (m, 2H), 0.37 (tt, J=9.3, 4.5 Hz, 1H), 0.20-0.12 (m, 1H).

### Int-16B

### (E)-ethyl 2-((2S,6R)-2-cyclopropyl-6-methyldihydro-2H-pyran-4(3H)-ylidene)-2-formamidoacetate

LC/MS retention time 1.29 min; m/z 268.2 (MH+). LC data was recorded on a Shimadzu chromatograph equipped with a Waters Aquity BEH C18 2.1×50 mm, 1.7 μm particles. The elution conditions employed a flow rate of 0.8 mL/min, a gradient of 70% solvent A/30% solvent B to 50% solvent A/50% solvent B, a gradient time of 1.5 min, a hold time of 0.5 min, and an analysis time of 2 min where solvent A was 10% MeOH/90% H₂O/0.1% TFA and solvent B was 10% H₂O/90% MeOH/0.1% TFA. MS data was determined using a Micromass Platform for LC in electrospray mode. ¹H NMR (500 MHz, CDCl₃) δ 8.26 (d, J=1.3 Hz, 0.7H), 7.99 (d, J=11.3 Hz, 0.3H), 6.80 (br. s., 0.7H), 6.66 (d, J=11.2 Hz, 0.3H), 4.33-4.23 (m, 2H), 4.22-4.11 (m, 1H), 3.21 (dd, J=14.0, 5.5 Hz, 0.7H), 3.16-3.03 (m, 1.6H), 2.93 (dd, J=14.1, 4.0 Hz, 0.7H), 2.67 (dd, J=14.0, 3.9 Hz, 0.3H), 2.49 (dd, J=14.0, 3.5 Hz, 0.7H), 2.23 (dd, J=14.0, 7.6 Hz, 0.3H), 2.09 (dd, J=14.0, 8.4 Hz, 0.7H), 1.39-1.31 (m, 3H), 1.25-1.20 (m, 3H), 1.19-1.10 (m, 0.7H), 1.06-0.98 (m, 0.3H), 0.62-0.53 (m, 2H), 0.45-0.36 (m, 1H), 0.28-0.18 (m, 1H).

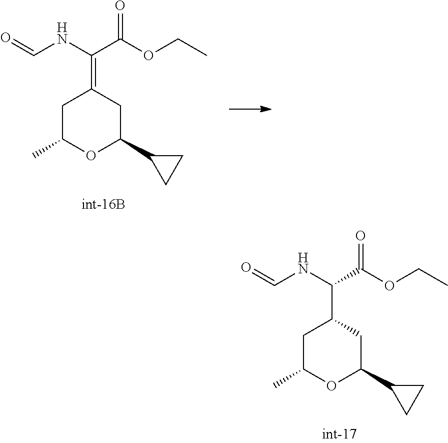 [SMILES: CCOC(=O)/C(NC=O)=C1/C[C@@H](C)O[C@H](C2CC2)C1 ; CCOC(=O)[C@@H](NC=O)[C@H]1C[C@@H](C)O[C@H](C2CC2)C1]

### Int-17

### (S)-ethyl 2-((2S,4S,6R)-2-cyclopropyl-6-methyltetrahydro-2H-pyran-4-yl)-2-formamidoacetate

In a Parr pressure vessel, (E)-ethyl 2-((2S,6R)-2-cyclopropyl-6-methyldihydro-2H-pyran-4(3H)-ylidene)-2-formamidoacetate (1.41 g, 5.27 mmol) was dissolved into MeOH (50 mL) and nitrogen was bubbled through the reaction solution for 10 min. Then (S,S)-Me-BPE-Rh (0.073 g, 0.132 mmol) was added, the nitrogen bubbling continued for 2 min and then the vessel was placed onto a Parr hydrogenator. The reaction was vacuum flushed with nitrogen (4×) and then with hydrogen (4×) and shaken under hydrogen (60 psi) for 2 days. The reaction was concentrated and then purified using a Biotage Horizon (40 g SiO₂, 50-75% EtOAc/hexanes) to yield (S)-ethyl 2-((2S,4S,6R)-2-cyclopropyl-6-methyltetrahydro-2H-pyran-4-yl)-2-formamidoacetate (1.356 g) as a clear colorless viscous oil. LC/MS retention time 1.16 min; m/z 270.2 (MH+). LC data was recorded on a Shimadzu LC-10AS liquid chromatograph equipped with a Phenomenex-Luna 3 μm particles., C18 2.0×30 mm column using a SPD-10AV UV-Vis detector at a detector wave length of 220 nM. The elution conditions employed a flow rate of 1 mL/min, a gradient of 100% solvent A/0% solvent B to 0% solvent A/100% solvent B, a gradient time of 2 min, a hold time of 1 min, and an analysis time of 3 min where solvent A was 10% acetonitrile/90% H₂O/0.1% TFA and solvent B was 10% H₂O/90% acetonitrile/0.1% TFA. MS data was determined using a Micromass Platform for LC in electrospray mode. ¹H NMR (400 MHz, CDCl₃) (10:1 mixture of amide rotamers, only the major rotamer reported) δ 8.29 (s, 1H), 6.14 (d, J=8.0 Hz, 1H), 4.72 (dd, J=9.0, 5.0 Hz, 1H), 4.37-4.17 (m, 2H), 3.94-3.84 (m, 1H), 3.06-2.99 (m, 1H), 2.53-2.40 (m, 1H), 1.67 (d, J=13.1 Hz, 1H), 1.58-1.50 (m, 2H), 1.32 (t, J=7.0 Hz, 3H), 1.27 (t, J=7.0 Hz, 1H), 1.18 (d, J=6.0 Hz, 3H), 1.07 (q, J=12.5 Hz, 1H), 0.55 (dqd, J=12.9, 8.5, 4.4 Hz, 2H), 0.46-0.39 (m, 1H), 0.16-0.07 (m, 1H).

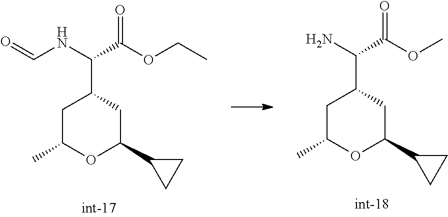 [SMILES: CCOC(=O)[C@@H](NC=O)[C@H]1C[C@@H](C)O[C@H](C2CC2)C1 ; COC(=O)[C@@H](N)[C@H]1C[C@@H](C)O[C@H](C2CC2)C1]

### Int-18

### (S)-ethyl 2-amino-2-((2S,4S,6R)-2-cyclopropyl-6-methyltetrahydro-2H-pyran-4-yl)acetate

A 1.5 N aq. HCl (7.86 mL, 11.79 mmol) solution was added to a solution of (S)-ethyl 2-((2S,4S,6R)-2-cyclopropyl-6-methyltetrahydro-2H-pyran-4-yl)-2-formamidoacetate (1.27 g, 4.72 mmol) in EtOH (16 mL) and the reaction mixture was stirred at 52° C. for 16 h. The reaction mixture was concentrated, and the residue azeotroped with EtOH to afford crude (S)-ethyl 2-amino-2-((2S,4S,6R)-2-cyclopropyl-6-methyltetrahydro-2H-pyran-4-yl)acetate as a white solid. This material was used in the next step without purification. LC/MS retention time 1.026 min; m/z 242.2 (MH+). LC data was recorded on a Shimadzu chromatograph equipped with a Waters Aquity BEH C18 2.1×50 mm, 1.7 μm particles. The elution conditions employed a flow rate of 0.8 mL/min, a gradient of 70% solvent A/30% solvent B to 50% solvent A/50% solvent B, a gradient time of 1.5 min, a hold time of 0.5 min, and an analysis time of 2 min where solvent A was 10% MeOH/90% H₂O/0.1% TFA and solvent B was 10% H₂O/90% MeOH/0.1% TFA. MS data was determined using a Micromass Platform for LC in electrospray mode.

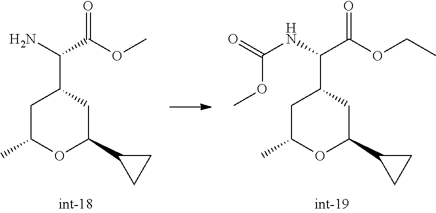 [SMILES: COC(=O)[C@@H](N)[C@H]1C[C@@H](C)O[C@H](C2CC2)C1 ; CCOC(=O)[C@@H](NC(=O)OC)[C@H]1C[C@@H](C)O[C@H](C2CC2)C1]

### Int-19

### (S)-ethyl 2-((2S,4S,6R)-2-cyclopropyl-6-methyltetrahydro-2H-pyran-4-yl)-2-((methoxycarbonyl)amino)acetate

Methyl chloroformate (0.55 mL, 7.1 mmol) was added to a solution of crude (S)-ethyl 2-amino-2-((2S,4S,6R)-2-cyclopropyl-6-methyltetrahydro-2H-pyran-4-yl)acetate (4.72 mmol) in DCM (30 mL) cooled to 0° C. DIPEA (2.47 mL, 14.2 mmol) was then added and the reaction mixture was allowed to warm to RT and stirred for 16 h. The reaction was concentrated and purified with a Biotage Horizon (80 g SiO₂, 0-50% EtOAc/hexanes) to yield (S)-ethyl 2-((2S,4S,6R)-2-cyclopropyl-6-methyltetrahydro-2H-pyran-4-yl)-2-((methoxycarbonyl)amino)acetate (1.21 g) as clear amber viscous oil. LC/MS retention time 1.30 min; m/z 300.2 (MH+). LC data was recorded on a Shimadzu chromatograph equipped with a Waters Aquity BEH C18 2.1×50 mm, 1.7 μm particles. The elution conditions employed a flow rate of 0.8 mL/min, a gradient of 70% solvent A/30% solvent B to 50% solvent A/50% solvent B, a gradient time of 1.5 min, a hold time of 0.5 min, and an analysis time of 2 min where solvent A was 10% MeOH/90% H₂O/0.1% TFA and solvent B was 10% H₂O/90% MeOH/0.1% TFA. MS data was determined using a Micromass Platform for LC in electrospray mode. ¹H NMR (500 MHz, CDCl₃) δ 5.24 (d, J=8.7 Hz, 1H), 4.36-4.24 (m, 2H), 4.20 (dq, J=10.8, 7.1 Hz, 1H), 3.91-3.84 (m, 1H), 3.70 (s, 3H), 3.06-2.99 (m, 1H), 2.45-2.35 (m, 1H), 1.64 (d, J=12.8 Hz, 1H), 1.57-1.48 (m, 2H), 1.30 (t, J=7.1 Hz, 3H), 1.26 (br. s., 1H), 1.17 (d, J=6.1 Hz, 3H), 1.14-1.04 (m, 1H), 0.60-0.49 (m, 2H), 0.44-0.38 (m, 1H), 0.13-0.07 (m, 1H).

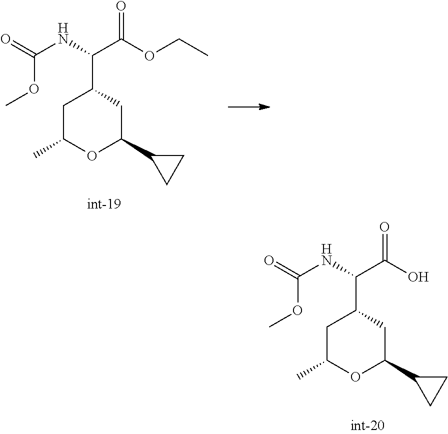 [SMILES: CCOC(=O)[C@@H](NC(=O)OC)[C@H]1C[C@@H](C)O[C@H](C2CC2)C1 ; COC(=O)N[C@H](C(=O)O)[C@H]1C[C@@H](C)O[C@H](C2CC2)C1]

### Int-20

### (S)-2-((2S,4S,6R)-2-cyclopropyl-6-methyltetrahydro-2H-pyran-4-yl)-2-((methoxycarbonyl)amino)acetic acid

(S)-Ethyl 2-((2S,4S,6R)-2-cyclopropyl-6-methyltetrahydro-2H-pyran-4-yl)-2-((methoxycarbonyl)amino)acetate (1.73 g, 5.49 mmol) was dissolved into THF (18 mL) and then treated with 1M aqueous LiOH (11.0 mL, 11.0 mmol) and the reaction mixture was stirred at RT for 16 h. The reaction was neutralized with 1M HCl (11 mL) and then extracted with EtOAc (3×20 mL). The combined organic component was washed with brine (20 mL), dried (MgSO₄), filtered and concentrated to a white solid which was dissolved into Et₂O (30 mL) and concentrated (3×) to yield (S)-2-((2S,4S,6R)-2-cyclopropyl-6-methyltetrahydro-2H-pyran-4-yl)-2-((methoxycarbonyl)amino)acetic acid (1.49 g) as a white solid. The material was used without further purification. LC/MS retention time 1.65 min; m/z 272.15 (MH+). LC data was recorded on a Shimadzu LC-10AS liquid chromatograph equipped with a Phenomenex-Luna 3 μm particles, C18 2.0×50 mm column using a SPD-10AV UV-Vis detector at a detector wave length of 220 nM. The elution conditions employed a flow rate of 0.8 mL/min, a gradient of 100% solvent A/0% solvent B to 0% solvent A/100% solvent B, a gradient time of 4 min, a hold time of 1 min, and an analysis time of 5 min where solvent A was 10% acetonitrile/90% H₂O/0.1% TFA and solvent B was 10% H₂O/90% acetonitrile/0.1% TFA. MS data was determined using a Micromass Platform for LC in electrospray mode. ¹H NMR (400 MHz, CDCl₃) δ 7.73 (br. s., 1H), 5.35 (d, J=8.8 Hz, 1H), 4.37 (dd, J=8.5, 4.8 Hz, 1H), 3.94 (dd, J=10.0, 5.8 Hz, 1H), 3.81-3.67 (m, 3H), 3.08 (dd, J=9.7, 4.6 Hz, 1H), 2.46 (d, J=3.3 Hz, 1H), 1.76-1.54 (m, 3H), 1.33-1.07 (m, 5H), 0.62-0.50 (m, 2H), 0.47-0.36 (m, 1H), 0.20-0.08 (m, 1H).

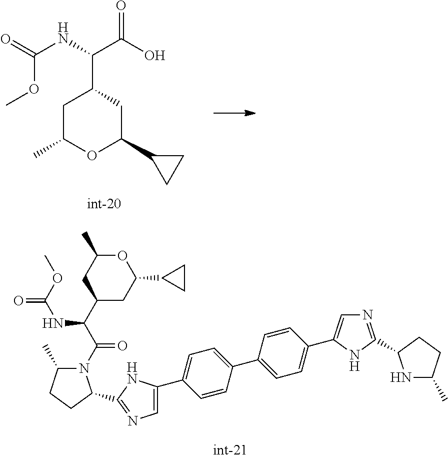 [SMILES: COC(=O)N[C@H](C(=O)O)[C@H]1C[C@@H](C)O[C@H](C2CC2)C1 ; COC(=O)N[C@H](C(=O)N1[C@@H](C)CC[C@H]1c1ncc(-c2ccc(-c3ccc(-c4cnc([C@@H]5CC[C@H](C)N5)[nH]4)cc3)cc2)[nH]1)[C@H]1C[C@@H](C)O[C@H](C2CC2)C1]

### Int-21

### methyl ((S)-1-((2S,4S,6R)-2-cyclopropyl-6-methyltetrahydro-2H-pyran-4-yl)-2-((2S,5S)-2-methyl-5-(5-(4′-(2-((2S,5S)-5-methylpyrrolidin-2-yl)-1H-imidazol-5-yl)-[1,1′-biphenyl]-4-yl)-1H-imidazol-2-yl)pyrrolidin-1-yl)-2-oxoethyl)carbamate

HATU (1.38 g, 3.61 mmol) was added to a solution of 4,4′-bis(2-((2S,5S)-5-methylpyrrolidin-2-yl)-1H-imidazol-5-yl)-1,1′-biphenyl, 4 HCl (1.44 g, 2.41 mmol) and (S)-2-((2S,4S,6R)-2-cyclopropyl-6-methyltetrahydro-2H-pyran-4-yl)-2-((methoxycarbonyl)amino)acetic acid (980 mg, 3.61 mmol) in DMF (12 mL) and DIPEA (3.36 mL, 19.3 mmol) and the reaction mixture was stirred under nitrogen at RT for 18 h. The reaction was concentrated and partitioned between water (40 mL) and EtOAc (50 mL). The aqueous component was further extracted with EtOAc (2×20 mL) and the combined organic component were washed with ½ sat. NaHCO₃ (˜40 mL), brine (˜40 mL) and then dried (MgSO₄) filtered and concentrated. The crude material was purified using a Biotage Horizon (80 g SiO₂, 1-5% MeOH/DCM followed by) to isolate the bis capped material dimethyl ((R,S,S,1S,1′S)-((2S,2S,5S,5′S)-5,5′-(5,5′-([1,1′-biphenyl]-4,4′-diyl)bis(1H-imidazole-5,2-diyl))bis(2-methylpyrrolidine-5,1-diyl))bis(1-((2S,4S,6R)-2-cyclopropyl-6-methyltetrahydro-2H-pyran-4-yl)-2-oxoethane-2,1-diyl))dicarbamate (1.37 g) as an off white solid. The column was further eluted (10% MeOH/DCM with 0.5% TEA) to isolate the mono capped material (title compound) methyl ((S)-1-((2S,4S,6R)-2-cyclopropyl-6-methyltetrahydro-2H-pyran-4-yl)-2-((2S,5S)-2-methyl-5-(5-(4′-(2-((2S,5S)-5-methylpyrrolidin-2-yl)-1H-imidazol-5-yl)-[1,1′-biphenyl]-4-yl)-1H-imidazol-2-yl)pyrrolidin-1-yl)-2-oxoethyl)carbamate (440 mg) as a yellow solid. LC/MS: Retention time 1.21 min; m/z 706.5 (MH+). Column: Waters BEH C18, 2.1×50 mm, 1.7 μm particles; Mobile Phase A: 10:90 MeOH:water with 0.1% TFA; Mobile Phase B: 90:10 MeOH:water with 0.1% TFA; Temperature: 40° C.; Gradient: 0% B, 0-100% B over 1.5 minutes, then a 0.5-minute hold at 100% B; Flow: 0.8 mL/min; Detection: UV at 220 nm.

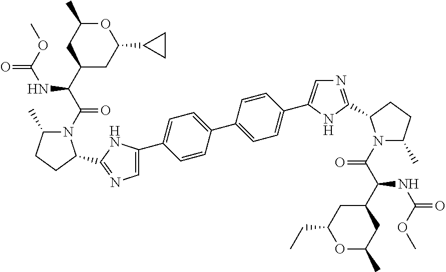 [SMILES: CC[C@@H]1C[C@@H]([C@H](NC(=O)OC)C(=O)N2[C@@H](C)CC[C@H]2c2ncc(-c3ccc(-c4ccc(-c5cnc([C@@H]6CC[C@H](C)N6C(=O)[C@@H](NC(=O)OC)[C@H]6C[C@@H](C)O[C@H](C7CC7)C6)[nH]5)cc4)cc3)[nH]2)C[C@@H](C)O1]

### EXAMPLE-1

### methyl N-[(1S)-1-[(2S,4S,6R)-2-cyclopropyl-6-methyloxan-4-yl]-2-[(2S,5S)-2-{5-[4-(4-{2-[(2S,5S)-1-[(2S)-2-[(2R,4S,6R)-2-ethyl-6-methyloxan-4-yl]-2-[(methoxycarbonyl)amino]acetyl]-5-methylpyrrolidin-2-yl]-1H-imidazol-5-yl}phenyl)phenyl]-1H-imidazol-2-yl}-5-methylpyrrolidin-1-yl]-2-oxoethyl]carbamate

HATU (15 mg, 0.039 mmol) was added to a stirred solution of methyl ((S)-1-((2S,4S,6R)-2-cyclopropyl-6-methyltetrahydro-2H-pyran-4-yl)-2-((2S,5S)-2-methyl-5-(5-(4′-(2-((2S,5S)-5-methylpyrrolidin-2-yl)-1H-imidazol-5-yl)-[1,1′-biphenyl]-4-yl)-1H-imidazol-2-yl)pyrrolidin-1-yl)-2-oxoethyl)carbamate (27 mg, 0.039 mmol) and (S)-2-((2R,4S,6R)-2-ethyl-6-methyltetrahydro-2H-pyran-4-yl)-2-((methoxycarbonyl)amino)acetic acid (10 mg, 0.039 mmol) in DMF (0.5 mL) and DIPEA (0.013 mL, 0.077 mmol) and the reaction mixture was stirred at RT for 16 h. The crude material was purified via preparative LC/MS with the following conditions:

Column: XBridge C18, 19×200 mm, 5-μm particles;
Mobile Phase A: 5:95 acetonitrile: water with 10-mM ammonium acetate;
Mobile Phase B: 95:5 acetonitrile:water with 10-mM ammonium acetate;
Gradient: 45-85% B over 15 minutes, then a 5-minute hold at 100% B;
Flow: 20 mL/min.
Yielded 18.5 mg of the title compound. LC/MS: Retention time 1.99 min; m/z 947.9 (MH+). Column: Waters BEH C18, 2.0×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B, 0-100% B over 3 minutes, then a 0.5-minute hold at 100% B; Flow: 1 mL/min; Detection: UV at 220 nm.

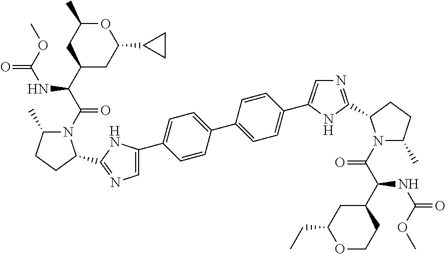 [SMILES: CC[C@@H]1C[C@@H]([C@H](NC(=O)OC)C(=O)N2[C@@H](C)CC[C@H]2c2ncc(-c3ccc(-c4ccc(-c5cnc([C@@H]6CC[C@H](C)N6C(=O)[C@@H](NC(=O)OC)[C@H]6C[C@@H](C)O[C@H](C7CC7)C6)[nH]5)cc4)cc3)[nH]2)CCO1]

### EXAMPLE-2

### methyl N-[(1S)-1-[(2S,4S,6R)-2-cyclopropyl-6-methyloxan-4-yl]-2-[(2S,5S)-2-{5-[4-(4-{2-[(2S,5S)-1-[(2S)-2-[(2R,4S)-2-ethyloxan-4-yl]-2-[(methoxycarbonyl)amino]acetyl]-5-methylpyrrolidin-2-yl]-1H-imidazol-5-yl}phenyl)phenyl]-1H-imidazol-2-yl}-5-methylpyrrolidin-1-yl]-2-oxoethyl]carbamate

HATU (16 mg, 0.041 mmol) was added to a solution of methyl ((S)-1-((2S,4S,6R)-2-cyclopropyl-6-methyltetrahydro-2H-pyran-4-yl)-2-((2S,5S)-2-methyl-5-(5-(4′-(2-((2S,5S)-5-methylpyrrolidin-2-yl)-1H-imidazol-5-yl)-[1,1′-biphenyl]-4-yl)-1H-imidazol-2-yl)pyrrolidin-1-yl)-2-oxoethyl)carbamate (29 mg, 0.041 mmol) and (S)-2-((2R,4S)-2-ethyltetrahydro-2H-pyran-4-yl)-2-((methoxycarbonyl)amino)acetic acid (10 mg, 0.041 mmol) in DMF (0.5 mL) and DIPEA (0.014 mL, 0.082 mmol) and the reaction mixture was stirred at RT for 16 h. The crude material was purified via preparative LC/MS with the following conditions:

Column: XBridge C18, 19×200 mm, 5-μm particles;
Mobile Phase A: 5:95 acetonitrile:water with 10-mM ammonium acetate;
Mobile Phase B: 95:5 acetonitrile:water with 10-mM ammonium acetate;
Gradient: 45-85% B over 15 minutes, then a 5-minute hold at 100% B;
Flow: 20 mL/min.
Yielded 13.2 mg of the title compound. LC/MS: Retention time 1.93 min; m/z 933.9 (MH+). Column: Waters BEH C18, 2.0×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B, 0-100% B over 3 minutes, then a 0.5-minute hold at 100% B; Flow: 1 mL/min; Detection: UV at 220 nm.

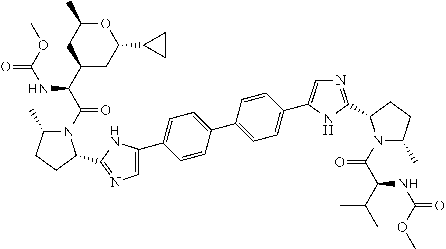 [SMILES: COC(=O)N[C@H](C(=O)N1[C@@H](C)CC[C@H]1c1ncc(-c2ccc(-c3ccc(-c4cnc([C@@H]5CC[C@H](C)N5C(=O)[C@@H](NC(=O)OC)[C@H]5C[C@@H](C)O[C@H](C6CC6)C5)[nH]4)cc3)cc2)[nH]1)C(C)C]

### EXAMPLE-3

### methyl N-[(1S)-1-[(2S,4S,6R)-2-cyclopropyl-6-methyloxan-4-yl]-2-[(2S,5S)-2-{5-[4-(4-{2-[(2S,5S)-1-[(2S)-2-[(methoxycarbonyl)amino]-3-methylbutanoyl]-5-methylpyrrolidin-2-yl]-1H-imidazol-5-yl}phenyl)phenyl]-1H-imidazol-2-yl}-5-methylpyrrolidin-1-yl]-2-oxoethyl]carbamate

HATU (41 mg, 0.11 mmol) was added to a stirred solution of methyl ((S)-1-((2S,4S,6R)-2-cyclopropyl-6-methyltetrahydro-2H-pyran-4-yl)-2-((2S,5S)-2-methyl-5-(5-(4′-(2-((2S,5S)-5-methylpyrrolidin-2-yl)-1H-imidazol-5-yl)-[1,1′-biphenyl]-4-yl)-1H-imidazol-2-yl)pyrrolidin-1-yl)-2-oxoethyl)carbamate (51 mg, 0.072 mmol) and (S)-2-((methoxycarbonyl)amino)-3-methylbutanoic acid (19 mg, 0.11 mmol) in DMF (0.7 mL) and DIPEA (0.038 mL, 0.22 mmol) and the reaction mixture was stirred under nitrogen for 3 h. One drop of 28-30% NH₄OH (aq.) was added and then the reaction was concentrated under a stream of nitrogen ON. The residual material was dissolved into MeOH (˜1 mL) filtered and purified via preparative LC/MS with the following conditions:

Column: XBridge C18, 19×200 mm, 5-μm particles;
Mobile Phase A: 5:95 acetonitrile:water with 10-mM ammonium acetate;
Mobile Phase B: 95:5 acetonitrile:water with 10-mM ammonium acetate;
Gradient: 45-85% B over 15 minutes, then a 5-minute hold at 100% B;
Flow: 20 mL/min.
Yielded 37.7 mg of the title compound. LC/MS: Retention time 1.95 min. m/z 863.7 (MH+). Column: Waters BEH C18, 2.0×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B, 0-100% B over 3 minutes, then a 0.5-minute hold at 100% B; Flow: 1 mL/min; Detection: UV at 220 nm.

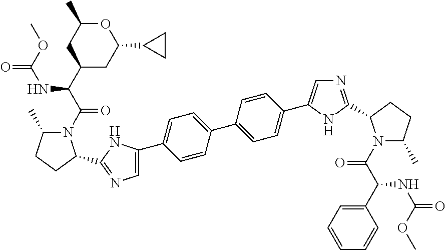 [SMILES: COC(=O)N[C@H](C(=O)N1[C@@H](C)CC[C@H]1c1ncc(-c2ccc(-c3ccc(-c4cnc([C@@H]5CC[C@H](C)N5C(=O)[C@H](NC(=O)OC)c5ccccc5)[nH]4)cc3)cc2)[nH]1)[C@H]1C[C@@H](C)O[C@H](C2CC2)C1]

### EXAMPLE-4

### methyl N-[(1S)-1-[(2S,4S,6R)-2-cyclopropyl-6-methyloxan-4-yl]-2-[(2S,5S)-2-{5-[4-(4-{2-[(2S,5S)-1-[(2R)-2-[(methoxycarbonyl)amino]-2-phenylacetyl]-5-methylpyrrolidin-2-yl]-1H-imidazol-5-yl}phenyl)phenyl]-1H-imidazol-2-yl}-5-methylpyrrolidin-1-yl]-2-oxoethyl]carbamate

HATU (40 mg, 0.10 mmol) was added to a stirred solution of methyl ((S)-1-((2S,4S,6R)-2-cyclopropyl-6-methyltetrahydro-2H-pyran-4-yl)-2-((2S,5S)-2-methyl-5-(5-(4′-(2-((2S,5S)-5-methylpyrrolidin-2-yl)-1H-imidazol-5-yl)-[1,1′-biphenyl]-4-yl)-1H-imidazol-2-yl)pyrrolidin-1-yl)-2-oxoethyl)carbamate (49 mg, 0.069 mmol) and (R)-2-((methoxycarbonyl)amino)-2-phenylacetic acid (21.78 mg, 0.104 mmol) in DMF (0.7 mL) and DIPEA (0.036 mL, 0.21 mmol) and the reaction mixture was stirred under nitrogen for 3 h. One drop of 28-30% NH₄OH (aq.) was added and then the reaction was concentrated under a stream of nitrogen ON. The residual material was dissolved into MeOH (˜1 mL) filtered and purified via preparative LC/MS with the following conditions:

Column: XBridge C18, 19×200 mm, 5-μm particles;
Mobile Phase A: 5:95 acetonitrile:water with 10-mM ammonium acetate;
Mobile Phase B: 95:5 acetonitrile:water with 10-mM ammonium acetate;
Gradient: 30-100% B over 13 minutes, then a 5-minute hold at 100% B;
Flow: 20 mL/min.
Yielded 36.7 mg of the title compound. LC/MS: Retention time 1.99 min. m/z 897.8 (MH+). Column: Waters BEH C18, 2.0×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B, 0-100% B over 3 minutes, then a 0.5-minute hold at 100% B; Flow: 1 mL/min; Detection: UV at 220 nm.

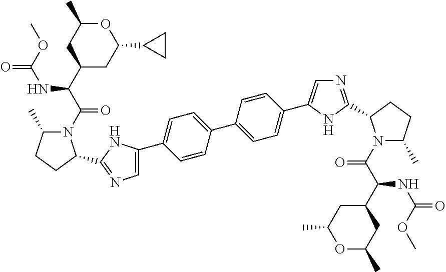

### EXAMPLE-5

### methyl N-[(1S)-1-[(2S,4S,6R)-2-cyclopropyl-6-methyloxan-4-yl]-2-[(2S,5S)-2-{5-[4-(4-{2-[(2S,5S)-1-[(2S)-2-[(2R,6R)-2,6-dimethyloxan-4-yl]-2-[(methoxycarbonyl)amino]acetyl]-5-methylpyrrolidin-2-yl]-1H-imidazol-5-yl}phenyl)phenyl]-1H-imidazol-2-yl}-5-methylpyrrolidin-1-yl]-2-oxoethyl]carbamate

HATU (41 mg, 0.11 mmol) was added to a stirred solution of methyl ((S)-1-((2S,4S,6R)-2-cyclopropyl-6-methyltetrahydro-2H-pyran-4-yl)-2-((2S,5S)-2-methyl-5-(5-(4′-(2-((2S,5S)-5-methylpyrrolidin-2-yl)-1H-imidazol-5-yl)-[1,1′-biphenyl]-4-yl)-1H-imidazol-2-yl)pyrrolidin-1-yl)-2-oxoethyl)carbamate (51 mg, 0.072 mmol) and (S)-2-((2R,6R)-2,6-dimethyltetrahydro-2H-pyran-4-yl)-2-((methoxycarbonyl)amino)acetic acid (27 mg, 0.11 mmol) in DMF (0.7 mL) and DIPEA (0.038 mL, 0.22 mmol) and the reaction mixture was stirred under nitrogen for 3 h. One drop of 28-30% NH₄OH (aq.) was added and then the reaction was concentrated under a stream of nitrogen ON. The residual material was dissolved into MeOH (˜1 mL) filtered and purified via preparative LC/MS with the following conditions:

Column: XBridge C18, 19×200 mm, 5-μm particles;
Mobile Phase A: 5:95 acetonitrile:water with 10-mM ammonium acetate;
Mobile Phase B: 95:5 acetonitrile:water with 10-mM ammonium acetate;
Gradient: 40-100% B over 15 minutes, then a 6-minute hold at 100% B;
Flow: 20 mL/min.
Yielded 39.9 mg of the title compound. LC/MS: Retention time 1.90 min; m/z 933.8 (MH+). Column: Waters BEH C18, 2.0×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B, 0-100% B over 3 minutes, then a 0.5-minute hold at 100% B; Flow: 1 mL/min; Detection: UV at 220 nm.

### Biological Activity

An HCV Replicon assay was utilized in the present disclosure, and was prepared, conducted and validated as described in commonly owned PCT/US2006/022197 and in O'Boyle et. al. Antimicrob Agents Chemother. 2005 April; 49(4):1346-53. Assay methods incorporating luciferase reporters have also been used as described (Apath.com).

HCV-neo replicon cells and replicon cells containing resistance substitutions in the NS5A region were used to test the currently described family of compounds. The compounds were determined to have differing degrees of reduced inhibitory activity on cells containing mutations vs. the corresponding inhibitory potency against wild-type cells. Thus, the compounds of the present disclosure can be effective in inhibiting the function of the HCV NS5A protein and are understood to be as effective in combinations as previously described in application PCT/US2006/022197 and commonly owned WO/04014852. It should be understood that the compounds of the present disclosure can inhibit multiple genotypes of HCV. Table 2 shows the EC₅₀ (Effective 50% inhibitory concentration) values of representative compounds of the present disclosure against the HCV 1b genotype.

The compounds of the present disclosure may inhibit HCV by mechanisms in addition to or other than NS5A inhibition. In one embodiment the compounds of the present disclosure inhibit HCV replicon and in another embodiment the compounds of the present disclosure inhibit NS5A. Compounds of the present disclosure may inhibit multiple genotypes of HCV. EC₅₀ ranges for all compounds are listed as A, meaning between 2 and 8 pM.

### Biology Table:

| Example | 1b LE (EC50, μM) | 1a LE (EC50, μM) |
|---|---|---|
| 1 | A | A |
| 2 | 5.76E−06 | 4.52E−06 |
| 3 | 2.72E−06 | 7.18E−06 |
| 4 | A | A |
| 5 | A | A |

The compounds of the present disclosure may inhibit HCV by mechanisms in addition to or other than NS5A inhibition. In one embodiment the compounds of the present disclosure inhibit HCV replicon and in another embodiment the compounds of the present disclosure inhibit NS5A. Compounds of the present disclosure may inhibit multiple genotypes of HCV containing multiple variants of NS5A sequences.

What is claimed is:

1. A compound of formula (I) 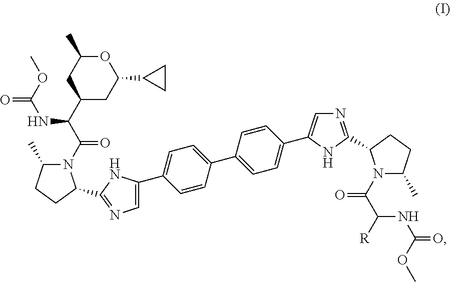
or a pharmaceutically acceptable salt thereof, wherein:
R is selected from isopropyl, phenyl, and 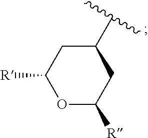
wherein R′ is selected from ethyl and methyl; and
R″ is selected from hydrogen and methyl.

2. A compound selected from 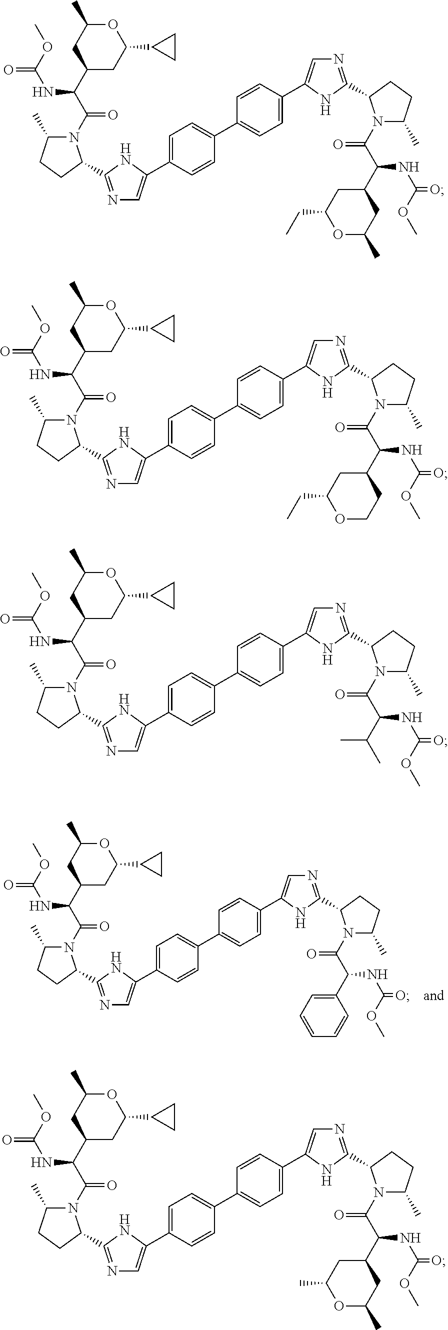
or a pharmaceutically acceptable salt thereof.

3. A composition comprising a compound of claim 1, or a pharmaceutically acceptable salt thereof, and a pharmaceutically acceptable carrier.

4. The composition of claim 3 further comprising one, two, or three additional compounds having anti-HCV activity.

5. The composition of claim 4 wherein at least one of the additional compounds is an interferon or a ribavirin.

6. The composition of claim 5 wherein the interferon is selected from interferon alpha 2B, pegylated interferon alpha, consensus interferon, interferon alpha 2A, interferon lambda, and lymphoblastoid interferon tau.

7. The composition of claim 4 wherein at least one of the additional compounds is selected from interleukin 2, interleukin 6, interleukin 12, a compound that enhances the development of a type 1 helper T cell response, interfering RNA, anti-sense RNA, Imiquimod, ribavirin, an inosine 5′-monophosphate dehydrogenase inhibitor, amantadine, and rimantadine.

8. The composition of claim 4 wherein at least one of the additional compounds is effective to inhibit the function of a target selected from HCV metalloprotease, HCV serine protease, HCV polymerase, HCV helicase, HCV NS4B protein, HCV entry, HCV assembly, HCV egress, HCV NS5A protein, and IMPDH for the treatment of an HCV infection.

9. A method of treating an HCV infection in a patient, comprising administering to the patient a therapeutically effective amount of a compound of claim 1, or a pharmaceutically acceptable salt thereof.

10. The method of claim 9 further comprising administering one, two, or three additional compounds having anti-HCV activity prior to, after or simultaneously with the compound of claim 1, or a pharmaceutically acceptable salt thereof.

11. The method of claim 10 wherein at least one of the additional compounds is an interferon or a ribavirin.

12. The method of claim 11 wherein the interferon is selected from interferon alpha 2B, pegylated interferon alpha, consensus interferon, interferon alpha 2A, interferon lambda, and lymphoblastoid interferon tau.

13. The method of claim 10 wherein at least one of the additional compounds is selected from interleukin 2, interleukin 6, interleukin 12, a compound that enhances the development of a type 1 helper T cell response, interfering RNA, anti-sense RNA, Imiquimod, ribavirin, an inosine 5′-monophosphate dehydrogenase inhibitor, amantadine, and rimantadine.

14. The method of claim 10 wherein at least one of the additional compounds is effective to inhibit the function of a target selected from HCV metalloprotease, HCV serine protease, HCV polymerase, HCV helicase, HCV NS4B protein, HCV entry, HCV assembly, HCV egress, HCV NS5A protein, and IMPDH for the treatment of an HCV infection.
